(19) United States
(12) Patent Application Publication
(10) Pub. No.: US 2021/0085672 A1
(43) Pub. Date: Mar. 25, 2021

(54) ORAL TABLET FORMULATIONS

(71) Applicant: INHERIS BIOPHARMA, INC., San Francisco, CA (US)

(72) Inventors: Sindhuri Maddineni, Dublin, CA (US); Shailendra Mandge, Indore, Madhya, Pradesh (IN); Sourish Mukherjee, Burdwan, West Bengal (IN); Vinod Balakrishnan Nair, Bhandup (W), Mumbai, Maharashtra State (IN); Vijaya Srinivas Sekuboyina, Andhra Pradesh (IN); Praveen Gaddam, Karimnagar, Telangana (IN); Kevin J. Brodbeck, Hillsborough, CA (US); Ramakrishna Gadiraju, Foster City, CA (US); Xue Ge, El Cerrito, CA (US); Michael A. Eldon, Redwood City, CA (US); Aleksandrs Odinecs, San Carlos, CA (US); Satyanarayana Goda, Stockton, CA (US); Rajendra Tandale, Atwater, CA (US); Shiladitya Bhattacharya, Stockton, CA (US)

(21) Appl. No.: 16/633,998

(22) PCT Filed: Jul. 25, 2018
(86) PCT No.: PCT/US2018/043733

§ 371 Date: Jan. 24, 2020

(30) Foreign Application Priority Data
Jul. 27, 2017 (IN) 201711026744

(51) Int. Cl. A61K 31/485; A61K 9/20; A61K 9/00

(52) U.S. Cl. CPC A61K 31/485; A61K 9/0053; A61K 9/2054

(57) ABSTRACT

Described herein are solid, pharmaceutical compositions, dosage forms and methods of making and using the same, wherein the solid compositions comprise at least one high viscosity agent. The solid, high viscosity agent-comprising pharmaceutical compositions, when comprised of an opioid drug, can reduce the potential for abuse of such drug. The solid dosage forms are characterized by having a significantly reduced extractability of an opioid drug comprised therein upon contact of the dosage form with a solvent such as a typical household solvent. The solid dosage forms, following contact with a household solvent, such as an aqueous or alcoholic solvent, generate a high viscosity solution, thereby discouraging abuse of the resulting formulation via intravenous (IV) injection.

### CROSS REFERENCE TO RELATED APPLICATION(S)

This application claims the benefit of priority under 35 U.S.C. 119(a) to Indian Patent Application No. 201711026744, filed Jul. 27, 2017, which is incorporated by reference herein in its entirety.

### FIELD

This disclosure relates generally to pharmaceutical compositions, methods for preparing such compositions, as well as uses of the compositions, among other things. More particularly, described herein are oral pharmaceutical compositions, formulations and solid dosage forms having poor or reduced syringe-ability, such as compositions having a high viscosity in solution. The high viscosity of the compositions, when in solution, makes it difficult to extract the pharmaceutical drug using aqueous, or other media. The compositions, formulations and dosage forms are useful, for example, to reduce the abuse potential of drugs.

### BACKGROUND

Pain is one of the most common reasons people seek medical treatment (Institute of Medicine, 2011, Relieving Pain in America: A Blueprint for Transforming Prevention, Care, Education, and Research, Washington, D.C., The National Academies Press; and Harstall, Pain Clinical Updates X, 1-4 (2003)). An estimated 19 percent of the U.S. population, or 39.4 million people, are reported to suffer from persistent pain (Kennedy et al., Journal of Pain, 15(10):979-984 (October 2014)).

Opioids are considered to be one of the most effective therapeutic options for treatment of pain, with 270 million prescriptions written in the U.S. alone in 2013 (IMS, NSP, NPA, and Defined Health 2013 Estimates; and Melnikova, I, Pain Market, Nature Reviews Drug Discovery, 9:589-90 (August 2010)). According to the Centers for Disease Control and Prevention (CDC), 115 people in the United States die every day from an opioid overdose (CDC, National Center for Health Statistics: 2017, available at the website wonder.cdc.gov). In 2014, nearly two million Americans either abused or were dependent upon prescription opioid pain relievers (CDC statistics at cdc.gov/drugoverdose/opioids/prescribed.html). In the United States, costs related to prescription opioid abuse totaled about $55.7 billion in 2007, of which 46 percent was attributable to workplace costs (e.g., lost productivity), 45 percent to healthcare costs (e.g., abuse treatment), and 9 percent to criminal justice costs (CDC, Prescription Drug Overdose Data, available at the website cdc.gov/drugoverdose/data/overdose.html (last updated 16 Oct. 2015), citing Birnbaum et al., Pain Medicine, 2011, 12:657-667).

For many opioid analgesics, the solid oral dosage forms (e.g. tablets) are significantly less potent than parenteral dosage forms (e.g. for intravenous injection) or mucosal dosage forms (e.g. for nasal administration) and therefore may require a higher dose of the opioid analgesic. The rapid bioavailability and increased potency of parenteral dosage forms of opioid drugs offers an incentive for abuse, for example by altering or tampering with the solid oral dosage form in order to administer the opioid analgesic contained therein via a parenteral route. For example, a solid oral dosage form (e.g. tablet) may be crushed, ground, and/or dissolved and/or heated and dissolved in a household solvent such as water. The resulting altered dosage form may then be inhaled or injected.

Various techniques have been proposed to prevent tampering with solid oral dosage forms, but with limited success. Suggested approaches include the use of agents that increase the difficulty in altering the dosage form, agents that prevent extraction of the opioid analgesic, and the addition of abuse deterrent agents to the solid dosage form. Previously proposed abuse deterrents include irritants (e.g. capsaicin) or bittering agents to deter improper administration of the solid dosage form. Other deterrent formulations include an opioid antagonist (such as, e.g., naloxone) that is not effective or is sequestered when administered orally, but has profound antagonistic action when administered parenterally, or is released only upon alteration of the dosage form. It has further been proposed to include a gelling agent such as xanthan gum to create a gel or viscous solution upon dissolution of the solid dosage form in a solvent, to reduce syringe-ability and discourage parenteral administration of the resulting formulation.

Although some abuse-deterrent opioid formulations are available, the mechanisms built into these formulations can be subverted. Further, modification of the solid dosage form to include abuse deterrents may undesirably make the resulting dosage form unsuitable for oral delivery (e.g. irritating upon oral administration) or less effective (e.g. through the use of opioid antagonists or by reducing the bioavailability of the opioid analgesic).

Given that opioid analgesics can have a high potential risk for abuse, there remains a need for improved abuse-deterrent compositions, formulations, dosage forms, and treatment methods comprising opioid analgesics. There is further a need for abuse-deterrent compositions and dosage forms that maintain bioavailability of the active ingredient when administered orally. An opioid analgesic that provides clinically meaningful analgesia, reduced CNS side effects, and/or a reduced potential for abuse would fill an important unmet medical need. The present compositions, dosage forms, and methods described herein address at least these needs.

### SUMMARY

Described herein are compositions, formulations, dosage forms, and methods that can reduce the potential for abuse of drugs having an abuse potential, such as opioid drugs. Also described are compositions, formulations and methods to prepare abuse deterrent dosage forms. The formulations described herein may significantly reduce the ability to extract an opioid drug from a solid dosage form upon contact with an extraction solvent (e.g. an aqueous solution such as water or an aqueous alcohol solution). The formulations described herein may also reduce the syringe-ability of an opioid drug product following addition of solvent to the formulation (such as an aqueous solvent) by generating a high viscosity solution, thereby discouraging abuse via parenteral (e.g. intravenous injection) administration. In addition to having reduced syringe-ability following dissolution in a suitable solvent, the formulations described herein may also enable immediate, or near immediate, release of an opioid from the formulation following administration. That is to say, in one or more embodiments, the release kinetics of the opioid from the formulations provided herein are not substantially adversely affected by addition of the one or more reduced abuse potential components to the formulation when compared to the same or substantially the same formulation absent the one or more reduced potential components.

In one or more embodiments, provided are compositions comprising an opioid drug and a high viscosity agent, such that when the composition is dissolved in a solvent or solution such as an aqueous solution or an alcoholic solution, the resulting composition has a viscosity that prevents parenteral administration.

In a first aspect, provided herein is a solid composition comprising (i) an opioid drug that may be α-6-mPEGₙ-O-oxycodol, wherein n is an integer selected from 1 to 30, or a pharmaceutically acceptable salt thereof, and (ii) at least one high viscosity agent. The composition preferably has a viscosity at 25° C. that is unsuitable for parenteral administration when the composition is dissolved in a household solvent or solvent mixture such as an aqueous solvent mixture, an alcoholic solvent or solvent mixture or water. In some embodiments, the viscosity of the composition is about 5-200 cP at 25° C. in an aqueous solution. In some embodiments, the viscosity of the composition is selected from at least about 10 cP, at least about 25 cP, at least about 50 cP, at least about 60 cP, at least about 75 cP, at least about 100 cP, at least about 200 cP, at least about 250 cP, at least about 500 cP, at least about 1000 cP, at least about 1200 cP, at least about 1500 cP, and about 1200-1600 cP for a 1% w/v aqueous solution at 25° C.

In some embodiments, at least one of the at least one high viscosity agents is sodium carboxymethylcellulose (NaCMC). In one or more embodiments, the NaCMC has a degree of substitution selected from 0.65 to 1.45, 0.65 to 0.9, 0.80 to 0.95, 1.15 to 1.45, and at least about 0.65. In some embodiments, the NaCMC has a molecular weight of between 80,000 to 800,000 Da.

In some embodiments, the composition comprises an amount of the high viscosity agent (either each agent or the total amount of all high viscosity agents) selected from 2.5-25%, 5 15%, 5-10%, 5-12%, 7.5-25%, 7.5-15%, 7.5-10%, 10-25%, 10-15%, 10-12%, and 12-15% of the high viscosity agent(s) by weight. In some further embodiments, the high viscosity agent is ionized at a low pH, e.g., at a pH less than 4.0, and is unionized at a pH of 6.0-9.0.

In some embodiments, the composition comprises a single high viscosity agent.

In some embodiments, the opioid drug is α-6-mPEGₙ-O-oxycodol, wherein n is an integer selected from 1 to 10 (e.g., 1, 2, 3, 4, 5, 6, 7, 8, 9, or 10), or a pharmaceutically acceptable salt thereof.

In one or more embodiments, the opioid drug has a molecular weight of 390 to 786 g/mol. In some embodiments, the amount of the opioid drug in the composition is selected from 25-65% and 28-30% by weight of the composition.

In some embodiments, the composition forms a gel when dissolved in an aqueous solution or an alcohol solution.

In some embodiments, the compositions as described herein are suitable for use as an analgesic and/or for use in the treatment of pain.

In a second aspect, provided herein is a method of treating pain in a patient in need thereof. In some embodiments, the method comprises administering a therapeutic amount of a solid composition as described herein to the patient. In preferred embodiments, the composition is orally administered.

In a third aspect, provided herein is a solid dosage form comprising a solid composition as described herein. In some embodiments, the solid dosage form is an oral dosage form. In some embodiments, the solid dosage form is a tablet or a capsule. In some other embodiments, the solid dosage form comprises a coating.

In a fourth aspect, provided herein is a method of manufacturing a solid dosage form, wherein the method comprises mixing at least one opioid drug and at least one high viscosity agent; and forming the mixture into a solid dosage form. In preferred embodiments, the solid dosage form, when dissolved in an aqueous or alcohol solution or in water, has a viscosity that is unsuitable for parenteral administration.

Additional embodiments are set forth in the following description and claims.

### BRIEF DESCRIPTION OF THE DRAWINGS

FIG. 1 is a graph of the mean (±SEM) plasma concentration in ng/mL of an exemplary opioid drug for up to 72 hours post-administration for (i) a reference (low viscosity formulation), (ii) a formulation comprising 7.5% NaCMC, and (iii) a 10% CMC formulation as described in detail in Example 6.

FIG. 2 is a graph of the dissolution profiles (shown as percent drug released over time (min.)) of (i) a reference 200 mg α-6-mPEG6-O-oxycodol formulation (X), (ii) exemplary Formulation A1 (comprising 10% NaCMC) (▴), and (iii) exemplary Formulation A4 (comprising 7.5% NaCMC) (▪), as described in detail in Example 10.

### DETAILED DESCRIPTION

The following terminology will be used in accordance with the definitions described below.

As used herein, the singular forms “a,” “an,” and “the” include plural referents unless the context clearly dictates otherwise.

As used herein, the term “alkyl” refers to a hydrocarbon chain, typically ranging from about 1 to 20 atoms in length. Such hydrocarbon chains are preferably but not necessarily saturated and can be branched or straight chain, although typically straight chain is preferred. The term also includes cycloalkyl when three or more carbon atoms are referenced. Exemplary alkyl groups include methyl, ethyl, propyl, butyl, pentyl, 1-methylbutyl, 1-ethylpropyl, 3-methylpentyl, cyclopropyl, and the like.

As used herein, the term “alkenyl” refers to a hydrocarbon chain of 2 to 20 carbon atoms having at least one carbon-carbon double bond in the chain.

As used herein, the term “solvent” refers generally to a substance in which one or more substances are, at least to some extent, dissolved. As used herein, the term “household solvent” refers generally to a solvent commonly commercially available for residential use. Non-limiting examples of household solvents include, but are not limited to, aqueous solutions (e.g., a solution that comprises water), water, alcoholic solutions (e.g., a solution that comprises an alcohol), ethanol, methanol, isopropyl alcohol, acetone, dichloromethane, ethyl acetate, hexanes, and mixtures thereof. In some embodiments, the household solvent is water, ethanol, or a mixture thereof. In some embodiments, the household solvent is an aqueous solution or water. In some embodiments, the household solvent is an alcohol solution or ethanol. It will be appreciated that the discussion below with reference to one solvent such as water applies to each of the other solvents described herein unless noted or otherwise ascertained by context.

As used herein the term “substantially” or “essentially” means near total or nearly complete, such as, for example 95% of a given quantity, or 99% or greater of a given quantity.

The description herein may refer to “composition”, “solid composition”, “formulation”, “solid dosage form” and “solid oral dosage form”. It will be appreciated that reference to the compositional elements as described herein with reference to any of a “composition”, “solid composition”, “formulation”, “solid dosage form” and “solid oral dosage form” may apply to any one of or all of the foregoing.

The term “gel” as used herein typically refers to a semi-solid composed of a liquid component and a solid component, which may be a polymer. In general, the polymer forms a three-dimensional network by virtue of covalent or non-covalent bonding.

Where a range of values is provided, it is intended that each intervening value between the upper and lower limit of that range as well as any other recited intervening value in the recited range is encompassed therein. For example, if a range of 1 to 5 mg is recited, it is intended that 1 mg, 2 mg, 3 mg, 4 mg, and 5 mg are also explicitly disclosed, as well as the range of values greater than or equal to 1 mg and the range of values less than or equal to 5 mg.

The term “patient,” or “subject” as used herein refers to a living organism suffering from or prone to a condition that can be prevented or treated by administration of a compound or composition as provided herein, such as pain, and includes both humans and animals. Subjects include, but are not limited to, mammals (e.g., murines, simians, equines, bovines, porcines, canines, felines, and the like), and preferably are human.

Overview

The instant disclosure addresses at least some of the problems and issues described above. After several attempts, Applicants have discovered compositions and methods relating to solid dosage forms or compositions comprising an opioid drug, wherein the compositions provided herein provide both tamper-resistance and maintain the bioavailability of the opioid drug, e.g., when compared to the composition absent one or more tamper-resistant components comprised therein. In some embodiments, the dosage forms and compositions described herein are oral dosage forms that are useful as an analgesic. Thus, ideally, such analgesic dosage forms and compositions must be effective when administered orally. For example, exemplary advantageous formulations are those that are effective to release an opioid drug in the acidic environment of the stomach following administration (e.g., having a release profile that is a rapid release profile, e.g., one that is substantially unchanged from that of the formulation absent any one or more abuse-deterrent components), but is not readily syringe-able when dissolved or suspended in a typical household solvent.

The present disclosure also provides a solid dosage form that is suitable for oral administration of an opioid drug and provides tamper-resistance by reducing syringe-ability of the solid dosage form when dissolved or placed in a solvent. In one aspect, the composition, solid dosage form, and one or more related methods comprises an opioid drug and at least one high viscosity agent, the features of which are described in greater detail below.

High Viscosity Agents

As used herein, the term “high viscosity agent” refers to a component of a composition, formulation or solid dosage form that forms a high viscosity solution or gel when dissolved, either fully or partially, in a household solvent. The composition may comprise at least one high viscosity agent, more than one high viscosity agent or a single high viscosity agent.

High viscosity agents include, but are not limited to, the following, as well as combinations thereof, where molecular weights as provided below are typically in daltons:

poly(ethylene glycol) (“PEG”), for example PEG having a weight average molecular weight of about 3350 (“PEG 3350”); or any other suitable molecular weight PEG; poly(ethylene oxide)s (“PEO”), for example PEO having a weight average molecular weight of 900,000 (“PEO 900K”), 400,000 (“PEO 400K”), or 8,000,000 (“PEO 8 million”);

nonionic, high molecular weight water-soluble poly(ethylene oxide) polymer resins, for example, having weight average molecular weight from about 100,000 to about 8,000,000, for example having weight average molecular weight of about 8,000,000 (e.g., POLYOX™ WSR 308 sold by Dow Chemical), for example having weight average molecular weight of about 5,000,000 (e.g., POLYOX™ WSR Coagulant sold by Dow Chemical), and for example, having a weight average molecular weight of about 900,000 (e.g., POLYOX™ WSR 1105 sold by Dow Chemical);

hydroxypropyl methylcellulose (“HPMC”), for example METHOCEL™ DC2 (e.g., grades K100LV, K4M, and K100M sold by Colorcon;

xanthan gum such as XANTURAL® 75 sold by CP Kelco, or XANTURAL® 180 sold by CP Kelco (or any other suitable XANTURAL® xanthan gum products available from CP Kelco);

sodium alginate (e.g., PROTANAL® PH 6160, sold by FMC Biopolymer);

carrageenan, for example GELCARIN® GP 379 sold by FMC BioPolymer;

carboxymethyl cellulose (“CMC”), such as sodium carboxymethyl cellulose (“NaCMC”), such as, for example, those sold by Spectrum Chemical MFG Corp (carboxymethyl cellulose sodium, low viscosity, 10-50 centipoise (cP); carboxymethyl cellulose sodium, low viscosity; carboxymethyl cellulose calcium salt; carboxymethyl cellulose sodium, high viscosity, 1500-3000 cP); CMC, such as NaCMC sold as AQUALON® by Ashland (carboxymethyl cellulose sodium, high viscosity, 1000-2800 cP (1% solution); carboxymethyl cellulose sodium, high viscosity, 1500-3000 cP (1% solution); carboxymethyl cellulose sodium, high viscosity, 2500-6000 cP (1% solution); carboxymethyl cellulose sodium, medium viscosity, 400-800 cP (2% solution)) and carboxymethyl cellulose calcium;

croscarmellose sodium such as AC-DI-SOL® sold by FMC BioPolymer; cellulose gum, low viscosity, FCC sold by Spectrum Chemical;

hydroxypropyl cellulose (“HPC”), for example the nonionic water-soluble cellulose ether hydroxypropyl cellulose products KLUCEL™ JXF Pharm, KLUCEL™ MF Pharm, and KLUCEL™ MF sold by Ashland.

In one or more embodiments, the high viscosity agent is one or more of PEG 3350, PEO (e.g. POLYOX™ WSR 308, POLYOX™ WSR Coagulant, POLYOX™ WSR 1105, PEO 900K, PEO 400K, PEO (8 million), METHOCEL™ DC2, HPMC K100M, sodium alginate (e.g. PROTANAL® PH 6160), xanthan gum (e.g. XANTURAL® 75, XANTURAL® 180), carrageenan (e.g. GELCARIN® GP 379), NaCMC, HPC (e.g. KLUCEL™ JXF Pharm, KLUCEL™ MF Pharm), croscarmellose sodium (e.g. AC-DI-SOL®), NaCMC, ammonium or aluminum salts of carboxymethyl cellulose, or combinations of any two or more of the foregoing.

In some embodiments, the high viscosity agent is croscarmellose sodium (e.g. AC-DI-SOL®), NaCMC, xanthan gum, or a combination of any two or more of the foregoing. In some further embodiments, the high viscosity agent is NaCMC. In yet some other embodiments, the high viscosity agent is croscarmellose sodium (e.g. AC-DI-SOL®). In yet some other embodiments, the high viscosity agent comprises croscarmellose sodium (e.g. AC-DI-SOL®) and NaCMC. In yet some further embodiments, the high viscosity agent comprises NaCMC and xanthan gum. In some additional embodiments, the high viscosity agent comprises croscarmellose sodium (e.g. AC-DI-SOL®) and xanthan gum. In yet some further embodiments, the composition does not include xanthan gum. In even further embodiments, the composition does not include a pH-independent polymer as a high viscosity agent.

In some further embodiments, the high viscosity agent is a pH-independent polymer. Examples include, without limitation, polyethylene oxide, xanthan gum, HPMC, and/or HPC.

In some embodiments, the high viscosity agent is a pH-dependent polymer including, without limitation, NaCMC and/or sodium alginate.

In yet some further embodiments, the high viscosity agent is NaCMC (sodium carboxymethylcellulose). NaCMC is an anionic, water-soluble polymer derived from cellulose (a cellulose ether having carboxymethyl groups substituted at certain hydroxyl positions within each anhydroglucose subunit of the polymer). High viscosity NaCMC polymers are sold, for example, by AQUALON® (e.g., grade 7H or 7HXF).

In some embodiments, the NaCMC has a degree of carboxymethyl group substitution of about 0.65-1.45 (carboxymethyl groups per anhydroglucose unit). In other embodiments, the NaCMC has a substitution range (i.e., degree of substitution) of about 0.65-0.90, about 0.80-0.95, or about 1.15-1.45. In some embodiments, the NaCMC has a molecular weight of about 80,000-800,000 Da or of about 90,000-700,000 Da. In some embodiments, NaCMC has an apparent dissociation constant of 5×10⁻⁵. In general, a higher degree of substitution results in more rapid dissolution of the polymer, e.g., in water. Additionally, in general, a lower molecular weight NaCMC provides a faster rate of dissolution (e.g., in a solvent such as water).

NaCMC for use in the compositions herein may have a viscosity range for a 1% solution of NaCMC in distilled water at 25° C. of about 1,000-6,000 cP. In some embodiments, the NaCMC has a viscosity range (for a 1% solution in distilled water at 25° C.) of about 1,500-3,000 cP, 1,000-2,800 cP, 2,500-6,000 cp. In some embodiments, NaCMC has a viscosity range for a 1% solution in distilled water at 25° C. of about 1,000-3,000 cP, about 1,000-4,000 cP, about 1,000-5,000 cP, about 1,000-6,000 cP, about 1,500-3,000 cP, about 1,500-4,000 cP, about 1,500-5,000 cP, about 1,500-6,000 cP, about 2,000-3,000 cP, about 2,000-4,000 cP, about 2,000-5,000 cP, about 2,000-6,000 cP, 3,000-4,000 cP, about 3,000-5,000 cP, about 3,000-6,000 cP, about 4,000-5,000 cP, about 4,000-6,000 cP, or about 5,000-6,000 cP. In some embodiments, NaCMC has a viscosity range for a 2% solution in distilled water at 25° C. of about 100-3,100 cP. In yet some additional embodiments, NaCMC has a viscosity range for a 2% solution in distilled water at 25° C. of about 100-200 cP, about 200-800 cP, about 400-800 cP, about 1,500-3,100 cP or 25-50 cP. In some embodiments, NaCMC has a viscosity range for a 4% solution in distilled water at 25° C. of about 50-200 cP.

It will be appreciated that viscosity may be determined or measured using any suitable method as known in the art. In exemplary embodiments, viscosity is measured with a viscometer (e.g., a Brookfield LV viscometer).

In some embodiments, the NaCMC has a particle size selected from D(0.5) of about 60-250 μm. In some embodiments, the NaCMC has a particle size selected from D(0.9) of greater than about 140 μm. Particle size may be determined by any suitable method as known in the art. In exemplary embodiments, particle size is determined by laser diffraction and model fitting (e.g., using the Malvern Mastersizer 2000).

In some embodiments, the composition comprises a substituted cellulose as the sole high viscosity agent. In some more particular embodiments, the composition comprises NaCMC as the sole high viscosity agent. It will be appreciated that in some embodiments where the composition comprises NaCMC or another substituted cellulose as the sole high viscosity agent, other thickening agents may be included where the thickening agents are included in an amount that does not substantially affect the viscosity of the composition in a household solvent.

Properties of High Viscosity Agents

In certain embodiments, the high viscosity agent can preferentially thicken aqueous solutions to avoid or reduce the ability to extract an opioid drug by dissolution, filtration, syringing, and/or other extraction techniques. The high viscosity agent may also have limited thickening/higher solvency in lower pH environments (e.g., the stomach) to allow more rapid uptake of the opioid drug into the blood stream. Without being limited as to theory, it is believed that in these embodiments, the high viscosity agent wets out or “extends its arms”, when in favorable conditions such as at neutral pHs to provide a high level of thickening and extraction prevention, while at less favorable solvent conditions, such as at low pHs, the arms do not freely extend, and thus the opioid drug can be more readily released from the pharmaceutical composition under such conditions.

Preferred general properties of the high viscosity agent include one or more of: rapid hydration in water to form a gel; insolubility in common organic solvents such as ethanol, ether, and acetone; ease of processability, for example, when comprised in a formulation for granulation and tableting; chemical and physical compatibility with an opioid drug and a wide range of oral dosage form excipients; and/or reliability in supply, for example source and consistency in quality.

In certain embodiments, the high viscosity agent may have one or more of the following properties:

Degree of Substitution: the high viscosity agent can have a degree of substitution of from about 0.65 to 0.85, or about 0.7 to 0.8, or about 0.75; and/or

Dissociation Constant (pKa): the high viscosity agent can have a dissociation constant from about 4.0 to 5.0, or about 4.1 to 4.9, or about 4.2 to 4.8, or about 4.3 to 4.7, or about 4.4 to 4.6, or of about 4.3, 4.4, 4.5, or 4.6. Oral dosage forms (e.g., tablets) comprising high viscosity agents with dissociation constants in this range can impair the ability of an opiod drug to be readily extracted into a weak acid, such as, for example, citric acid or tartaric acid.

Solubility: the high viscosity agent can be insoluble in some solvents and soluble in others. For example, the high viscosity agent can be insoluble in some household solvents and soluble in other household solvents. As one example, the high viscosity agent can be insoluble in acetone, ethanol, ether, and/or toluene, while also being soluble in water, but imparting a high viscosity when dissolved in water. In this manner, abuse deterrence may be provided by several routes (e.g., insolubility in some solvents and high viscosity in others). Oral dosage forms (e.g., tablets) comprising high viscosity agents with these solubility characteristics may help to reduce the abuse potential of the oral tablet dosage forms via dissolution in alcohol or other organic components. It will be appreciated that discussion herein with reference to an “oral tablet dosage form” as used herein may equally apply to other oral dosage forms including, but not limited to, capsules, caplets, and the like.

Viscosity: the high viscosity agent can have a viscosity in aqueous 1% w/v solution of 5-2000 mPa s (5-2000 cP) at 25° C. For example, 1200-1600 mPa s (1200-1600 cP) for a 1% w/v aqueous solution at 25° C. An increase in concentration of the high viscosity agent in an oral tablet dosage form can result in an increase in aqueous solution viscosity. This can enable flexibility in terms of the ability to select over a wide range of concentrations of a high viscosity agent in an oral tablet dosage form. In some embodiments, the composition has a viscosity of at least about 5-1000 cP or greater in 1% aqueous solutions at 25° C. In some embodiments, the composition has a viscosity of about 5-500 cP, about 5-250 cP, about 5-200 cP, about 5-180 cP, about 5-185 cP, about 5-175 cP, about 5-150 cP, about 5-125 cP, about 5-100 cP, about 5-75 cP, about 5-50 cP, about 5-25 cP, about 5-20 cP, about 5-15 cP, or about 5-10 cP. In some embodiments, the composition has a viscosity of about 10-1000 cP or greater, about 10-200 cP, about 10-180 cP, about 10-185 cP, about 10-175 cP, about 10-150 cP, about 10-125 cP, about 10-100 cP, about 10-75 cP, about 10-50 cP, about 10-25 cP, about 10-20 cP, about 10-15 cP, about 15-1000 cP or greater, about 15-200 cP, about 15-180 cP, about 15-185 cP, about 15-175 cP, about 15-150 cP, about 15-125 cP, about 15-100 cP, about 15-75 cP, about 15-50 cP, about 15-25 cP, about 15-20 cP, about 20-1000 cP or greater, about 20-200 cP, about 20-180 cP, about 20-185 cP, about 20-175 cP, about 20-150 cP, about 20-125 cP, about 20-100 cP, about 20-75 cP, about 20-50 cP, about 20-25 cP, about 25-1000 cP or greater, about 25-200 cP, about 25-180 cP, about 25-185 cP, about 25-175 cP, about 25-150 cP, about 25-125 cP, about 25-100 cP, about 25-75 cP, about 25-50 cP, about 50-1000 cP or greater, about 50-200 cP, about 50-180 cP, about 50-185 cP, about 50-175 cP, about 50-150 cP, about 50-125 cP, about 50-100 cP, about 50-75 cP, about 75-1000 cP or greater, about 75-200 cP, about 75-180 cP, about 75-185 cP, about 75-175 cP, about 75-150 cP, about 75-125 cP, about 75-100 cP, about 100-1000 cP or greater, about 100-200 cP, about 100-180 cP, about 100-185 cP, about 100-175 cP, about 100-150 cP, about 125-1000 cP or greater, about 125-200 cP, about 125-180 cP, about 125-185 cP, about 125-175 cP, about 125-150 cP, about 150-1000 cP or greater, about 150-200 cP, about 150-180 cP, about 150-185 cP, about 150-175 cP, about 175-1000 cP or greater, about 175-200 cP, about 175-180 cP, about 175-185 cP, about 185-1000 cP or greater, about 185-200 cP, about 180-1000 cP or greater, about 180-200 cP, or about 200-2000 cP or greater. In preferred embodiments, the aqueous solution is water.

In some embodiments, the composition has a viscosity of at least about 5 cP, 10 cP, 15 cP, 20 cP, 25 cP, 30 cP, 40 cP, 50 cP, 60 cP, 65 cP, 75 cP, 100 cP, 125 cP, 150 cP, 175 cP, 180 cP, 185 cP, 200 cP, 250 cP or 500 cP. In some embodiments, the composition has a viscosity of up to about 5 cP, 10 cP, 15 cP, 20 cP, 25 cP, 30 cP, 40 cP, 50 cP, 60 cP, 65 cP, 75 cP, 100 cP, 125 cP, 150 cP, 175 cP, 180 cP, 185 cP, 200 cP, 250 cP or 500 cP.

In some embodiments, the viscosity of solutions comprising the high viscosity agent is at a maximum at neutral or near neutral pH (e.g. pHs of about 6.0-9.0, or from about 4.0 to 9.0).

pH Stability: aqueous solutions of the high viscosity agent can be stable over a wide pH range, for example, over a pH range of from about pH 3 to 12, or from about pH 4 to 10. This pH stability can enable compatibility of the high viscosity agent with nearly all pharmaceutically acceptable salts in an oral tablet dosage form. This compatibility enables compatibility of the high viscosity agent with a wide range of pharmaceutically acceptable excipients in an oral tablet dosage form. The pH may be measured by any suitable means known in the art. In some embodiments, the pH is measured at room temperature using a pH meter.

Hydration Rate: the high viscosity agent can rapidly hydrate in water. Such rapid hydration feature allows rapid formation of a high viscosity solution after dispersion of an oral tablet dosage form comprising the high viscosity agent in water or any other suitable aqueous solvent mixture.

The following table provides illustrative properties for the exemplary high viscosity agents, sodium carboxymethyl cellulose (“NaCMC”) and xanthan gum (“XG”):

| Property | Sodium Carboxymethyl Cellulose | Xanthan Gum |
|---|---|---|
| Dissociation | 4.3 | 4.6 |
| constant | | |
| (pKa) | | |
| Solubility | Practically insoluble in acetone, ethanol | Practically insoluble in ethanol |
| | (95%), ether, and toluene. Easily | and ether; soluble in cold or warm |
| | dispersed in water at all temperatures, | water. |
| | forming clear, colloidal solutions. | |
| Viscosity | Aqueous 1% w/v solutions with | 1200-1600 mPa s (1200-1600 cP) |
| | viscosities of 5-2000 mPa s (5-2000 cP) | for a 1% w/v aqueous solution at |
| | may be obtained at 25° C. An increase | 25° C. An increase in concentration |
| | in concentration results in an increase | results in an increase in aqueous |
| | in aqueous solution viscosity, | solution viscosity. |
| pH Stability | The viscosity of sodium carboxymethyl | Aqueous solutions are stable over |
| | cellulose solution reaches a maximum | a wide pH range of 3 to 12. |
| | and is most stable at a pH range of | Maximum stability at pH 4 to 10 |
| | 6.5-9.0 to 10. At pH lower than 6.0, | and temperatures of 10 to 60° C. |
| | the viscosity rapidly decreases to a | Compatible with nearly all salts. |
| | minimum of about 4.0. | |
| | Compatible with nearly all salts. | |
| Hydration | Generally exhibits quick hydration. | The most rapid and efficient |
| Rate | Hydration rate can depend on grade, | hydration occurs in water. |
| | concentration, and particle size. | Hydration rate can depend on |
| | | particle size. |

NaCMC has a unique mechanism of polymer swelling and drug release over a range of pHs. Without being limited as to theory, it is believed that at low pHs (e.g. a pH of less than about 4, or even less than about 1-2), the NaCMC is ionized, has low swelling and/or provides rapid release of active agent. It is also believed that at neutral pHs, the NaCMC is substantially unionized, exhibits high swelling and/or provides slow drug release. The terms “low swelling” and “high swelling” are used herein with reference to the understanding in the art for swelling ratio or degree. In some embodiments, low swelling refers to less than about 5%, 10%, 25% or 30% swelling when the solid composition is placed in a solution, such as a household solvent system as described herein, such as, for example, an aqueous or alcoholic solvent. In some embodiments, high swelling refers to at least about 50%, 100%, 150%, 200% or more swelling when the solid composition is placed in a solvent, This uniqueness of NaCMC is effective to provide the following properties to an oral tablet formulation: (1) faster dissolution at pH 1.2 (pH of gastric contents in a fasted state) allows rapid release of the active agent from the formulation; and (2) slower release of an active agent in water (i.e., at neutral pHs) helps to impair unwanted extraction of the active agent, to thereby deter potential abuse.

High Viscosity Agents in Formulations

The high viscosity agent may be provided as an intra-granular component of a solid dosage form, as an extra-granular component of a solid dosage form, or as both an intra-granular and as an extra-granular component of a solid dosage form. In one or more embodiments, the high viscosity agent is provided as an extra-granular component of a solid dosage form. In yet some other embodiments, the high viscosity agent is provided as an intra-granular component of a solid dosage form. In yet some further embodiments, the high viscosity agent is provided as both an extra-granular and an intra-granular component of a solid dosage form.

The amount of high viscosity agent present in a solid dosage form should be sufficient to form a high viscosity solution or gel when the tablet is dissolved, either fully or partially, in a household solvent. In some embodiments, the weight of the high viscosity agent is from about 2.5% to 50% of the total weight of a solid dosage form. In some other embodiments, the weight of the high viscosity agent is from about 2.5% to 25% of the total weight of a solid dosage form. In yet other embodiments, the weight of the high viscosity agent is from about 2.5% to 20% of the total weight of a solid dosage form. In other embodiments, the weight of the high viscosity agent is from about 2.5% to 15% of the total weight of a solid dosage form. In some additional embodiments, the weight of the high viscosity agent is from about 2.5% to 12.5%, about 2.5% to 12%, about 2.5% to 10%, about 2.5% to 7.5%, or about 2.5% to 5.0% of the total weight of a solid dosage form. In further embodiments, the weight of the high viscosity agent is from about 3.0% to 12% of the total weight of a solid dosage form. In yet other embodiments, the weight of the high viscosity agent is from about 3.5% to 11% of the total weight of a solid dosage form.

In some embodiments, the weight of the high viscosity agent is from about 8% to 25% of the total weight of a solid dosage form. In other embodiments, the weight of the high viscosity agent is from about 8% to 20% of the total weight of a solid dosage form. In yet further embodiments, the weight of the high viscosity agent is from about 8% to 15% of the total weight of a solid dosage form. In other embodiments, the weight of the high viscosity agent is from about 9% to 13% of the total weight of a solid dosage form. In some embodiments, the weight of the high viscosity agent is from about 10% to 13% of the total weight of a solid dosage form.

In further embodiments, the high viscosity agent is from about 5% to 50% of the total weight of the solid dosage form. In other embodiments, the high viscosity agent is from about 5% to 15%, about 5% to 12%, about 5% to 7.5%, or about 5% to 10% of the total weight of the solid dosage form.

In some embodiments, the high viscosity agent is from about 7% to 12% of the total weight of the solid dosage form.

In other particular embodiments, the high viscosity agent is about 7.5% the total weight of the solid dosage form. In some embodiments, the high viscosity agent is from about 7.5% to 50%, about 7.5% to about 25%, about 7.5% to 20%, about 7.5% to 15%, about 7.5% to 12.5%, about 7.5% to 12%, or about 7.5% to 10%, of the total weight of the solid dosage form.

In yet another embodiment, the high viscosity agent is about 8.5% the total weight of the solid dosage form.

In some embodiments, the solid dosage form comprises about 10% to 50%, about 10% to 25%, about 10% to 20%, about 10% to 15%, or about 10% to 12% of the total weight of the solid dosage form.

In some additional embodiments, the solid dosage form comprises at least about 2.5%, about 5%, about 7.5%, about 10%, about 12.5%, about 15%, about 20%, about 25%, or about 50% of the high viscosity agent.

Properties of Formulations Comprising High Viscosity Agents

In certain embodiments, formulations comprising a high viscosity agent may have certain advantageous properties which can enable the formulations to both treat pain with, and reduce the abuse potential of, an opioid drug. In certain embodiments, formulations comprising a high viscosity agent can have certain advantageous properties related to treatment and abuse potential, and also be easily processed into solid dosage forms, such as tablets. In certain embodiments, formulations comprising a high viscosity agent can have certain advantageous properties related to treatment and abuse potential, and also be comprised of compounds well-understood and accepted by bodies which regulate and supervise pharmaceuticals, such as the United States Food and Drug Administration (“FDA”).

In certain embodiments, formulations comprising a high viscosity agent may possess one or more of the following properties.

Rapid release of mu-opioid agonist drug: formulations, such as oral tablet formulations, comprising a high viscosity agent can rapidly release an opioid drug when taken orally. In certain embodiments, oral tablet formulations comprising a high viscosity agent can release at least 80-100 percent of the opioid drug within 60 minutes after oral administration. In certain embodiments, oral tablet formulations comprising a high viscosity agent can release at least about 50-85 percent of the opioid drug within 15 minutes after oral administration.

Processability: formulations, such as oral tablet formulations, comprising a high viscosity agent can in certain embodiments be easily processed by conventional processing techniques. In certain embodiments, formulations comprising a high viscosity agent can be easily formed into tablets for oral administration with minimal sticking to a tablet die. In certain embodiments, formulations comprising a high viscosity agent can be provided as a free flowing powder to enable useful mixing, blending, granulation, filling, and compression of the formulation.

Reduced syringe-ability: formulations, such as oral tablet formulations, comprising a high viscosity agent can, in certain embodiments, have reduced syringe-ability when compared to formulations not having (i.e., absent) a high viscosity agent. For example, a formulation without a high viscosity agent can have a syringe-ability (i.e., when placed in a suitable solvent as described herein) of about 66 to 79 weight percent while a formulation having a high viscosity agent can have a syringe-ability as low as about 0 to 37 weight percent, in certain embodiments, as low as about 0 to 4 weight percent, or about 3 to 12 weight percent, or about 20 to 37 weight percent, or about 5 to 28 weight percent. In some embodiments, the solid dosage forms described herein have a syringe-ability of about 0% to 50%, about 0% to 40%, about 0% to 35%, about 0% to 30%, about 0% to 25%, about 0% to 20%, about 0% to 15%, about 0% to 10%, or about 0% to 5%. In some embodiments, the solid dosage forms described herein have a syringe-ability of about 5% to 50%, about 5% to 40%, about 5% to 35%, about 5% to 30%, about 5% to 25%, about 5% to 20%, about 5% to 15%, about 5% to 10%, about 10% to 50%, about 10% to 40%, about 10% to 35%, about 10% to 30%, about 10% to 25%, about 10% to 20%, about 10% to 15%, about 15% to 50%, about 15% to 40%, about 15% to 35%, about 15% to 30%, about 15% to 25%, about 15% to 20%, about 20% to 50%, about 20% to 40%, about 20% to 35%, about 20% to 30%, about 20% to 25%, about 25% to 50%, about 25% to 40%, about 25% to 35%, about 25% to 30%, about 30% to 50%, about 30% to 40%, about 30% to 35%, about 35% to 50%, about 35% to 40%, or about 40% to 50%. In some embodiments, the solid oral dosage form comprising a high viscosity agent as described herein have a reduction in syringe-ability of at least about 5%, 10%, 15%, 20%, 25%, 30%, 40%, 50%, 60%, 70%, 75%, 80%, 90%, or 100% as compared to the same or similar formulation without the high viscosity agent.

It will be appreciated that syringe-ability may be measured according to any method known in the art. Some exemplary methods involve drawing or attempting to draw a solution or gel into a syringe and measuring the amount of solution drawn into a needle such as described in Example 7.

Reduced extraction of mu-opioid agonist drug: formulations, such as oral tablet formulations, comprising a high viscosity agent can, in certain embodiments, possess a reduced ability to extract the mu-opioid agonist drug from the formulation. For example, a formulation without a high viscosity agent may allow for recovery of about 67 to 77 weight percent of the mu-opioid agonist drug from the formulation, while a formulation comprising a suitable high viscosity agent may allow for recovery of about 10 to 45 weight percent of drug from the formulation, or about 0 weight percent from the formulation, or about 10 to 31 weight percent of the formulation, or less than about 10 weight percent from the formulation, or less than about 20 weight percent from the formulation.

Opioid Drugs

As used herein, the term “opioid drug” refers to a mu-opioid agonist analgesic compound.

In some embodiments, the opioid drug is selected from acetorphine, acetyldihydrocodeine, acetyldihydrocodeinone, acetylmorphinone, alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, etorphine, dihydroetorphine, fentanyl, heroin, hydrocodone, hydroxycodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphine, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propoxyphene, sufentanil, tilidine, tramadol, or a pharmaceutically acceptable salt any of the foregoing. In certain embodiments, the opioid drug is hydrocodone, morphine, hydromorphone, oxycodone, codeine, levorphanol, meperidine, methadone, oxymorphone, buprenorphine, fentanyl, dipipanone, heroin, tramadol, nalbuphine, etorphine, dihydroetorphine, butorphanol, or levorphanol, or a pharmaceutically acceptable salt thereof.

In some embodiments, the opioid drug is an alpha-6-mPEGₙ-O-oxycodol having the formula:

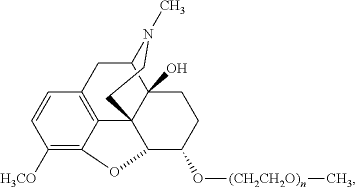

where n is an integer selected from 1-30 or a pharmaceutically acceptable salt thereof. In some embodiments, n is an integer selected from 1-10 (e.g., 1, 2, 3, 4, 5, 6, 7, 8, 9, and 10). Opioids such as alpha-6-mPEG₁₋₃₀-O-oxycodol, or e.g., alpha-6-mPEG₆-O-oxycodol, or a pharmaceutically acceptable salt form thereof, result in a relatively slow rate of entry into the central nervous system when compared to conventional opioids, independent upon dose level or route of administration. Moreover, such compounds are, in and of themselves, resistant to chemical and/or physical alteration to increase the rate of uptake into the brain. These opioids are capable of providing clinically meaningful analgesia in combination with reduced acute CNS-mediated side effects, such as euphoria, sedation, and respiratory depression, such that the formulations described herein are, in some embodiments, effective to provide yet an additional measure of protection against the possible extraction (i.e., removal) of the opioid from the solid oral dosage form, even given the suggested lower abuse potential of such compounds themselves when compared to classic opioid drugs such as oxycodone or fenanyl.

In one or more preferred embodiments, the opioid drug is alpha-6-mPEG₆-O-oxycodol, or a pharmaceutically acceptable salt thereof. In some embodiments, the drug is alpha-6-mPEG₆-O-oxycodol D-tartrate. In yet other embodiments, the drug is alpha-6-mPEG₆-O-oxycodol phosphate. Alpha-6-mPEG₆-O-oxycodol is a mu-opioid agonist analgesic.

In other embodiments, the opioid drug is a mu-opioid agonist according to Formula I:

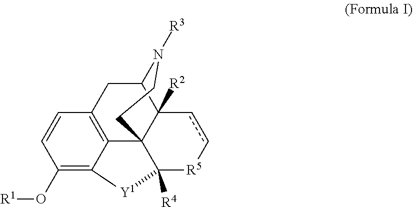

or a pharmaceutically acceptable salt, solvate, or prodrug thereof, wherein:

R¹ is hydrogen, —C(O)(C1-C10 alkyl), or C1-C10 alkyl;

R² is hydrogen or hydroxyl;

R³ is hydrogen or C1-C10 alkyl;

R⁴ is hydrogen or C1-C10 alkyl;

Y¹ is —O— or —S—;

R⁵ is —C(O)— or —CH(OR⁶)—;

R⁶ is hydrogen, C1-C10 alkyl, —C(O)(C1-C10 alkyl), or —(CH₂CH₂O)ₙE¹;

n is a positive integer selected over the range of 1 to 30;

E¹ is hydrogen, C1-C10 alkyl, or hydroxyl; and

the dotted line (---) represents an optional double bond.

In yet other embodiments, the opioid drug is a mu-opioid agonist according to Formula Ia or Formula Ib:

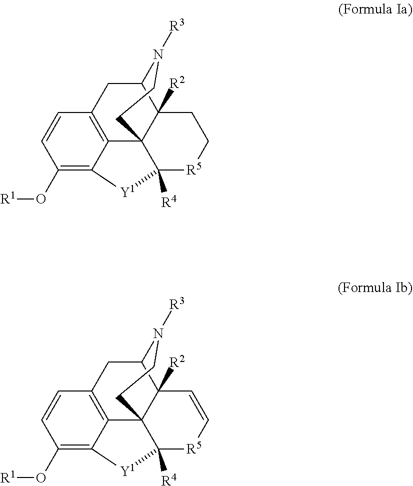

or a pharmaceutically acceptable salt, solvate, or prodrug thereof, wherein R¹, R², R³, R⁴, R⁵, and Y¹ are as described in the context of Formula I.

In one or more further embodiments, the opioid drug is a mu-opioid agonist according to Formula II:

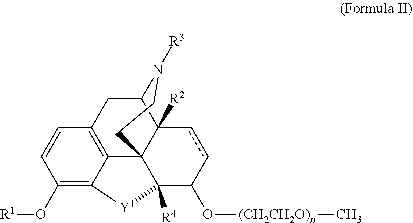

or a pharmaceutically acceptable salt, solvate, or prodrug thereof, wherein R¹, R², R³, R⁴, the dotted line, n, and Y¹ are as described in the context of Formula I.

In yet other embodiments, the opioid drug is a mu-opioid agonist according to Formula IIa or IIb:

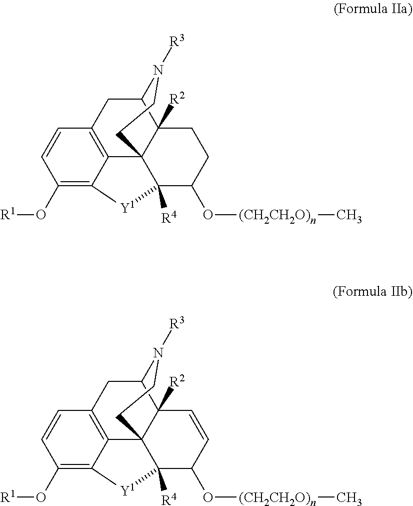

or a pharmaceutically acceptable salt, solvate, or prodrug thereof, wherein R¹, R², R³, R⁴, the dotted line, n, and Y¹ are as described in the context of Formula I.

In some embodiments, the opioid drug is a mu-opioid agonist according to Formula IIai, IIaii, IIbi, or IIbii:

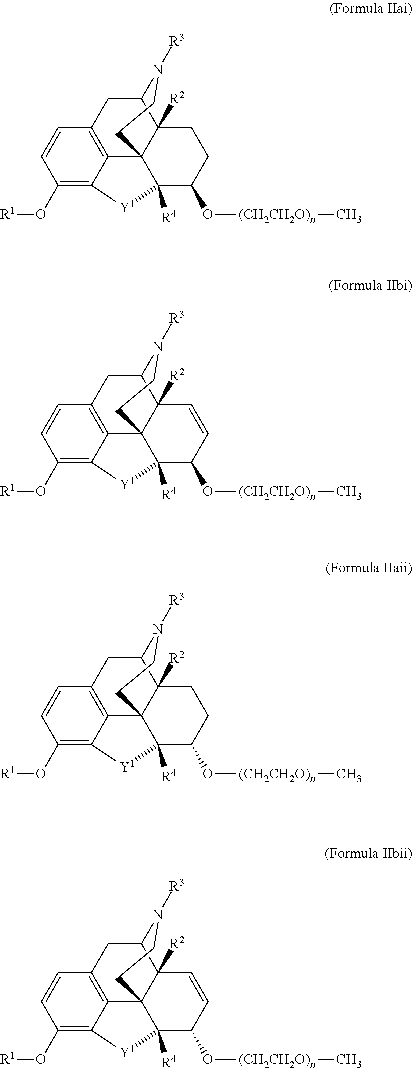

or a pharmaceutically acceptable salt, solvate, or prodrug thereof, wherein R¹, R², R³, R⁴, the dotted line, n, and Y¹ are as described in the context of Formula I.

In some particular embodiments, the opioid drug is a mu-opioid agonist according to any of Formulae I, Ia, Ib, II, IIa, IIb, IIai, IIaii, IIbi, or IIbii, wherein R¹ is methyl. In yet other embodiments, the opioid drug is a mu-opioid agonist according to any of Formulae I, Ia, Ib, II, IIa, IIb, IIai, IIaii, IIbi, or IIbii, wherein R¹ is ethyl. In further embodiments, the opioid drug is a mu-opioid agonist according to any of Formulae I, Ia, Ib, II, IIa, IIb, IIai, IIaii, IIbi, or IIbii, wherein R¹ is —C(O)CH₃.

In yet other embodiments, the opioid drug is a mu-opioid agonist according to any of Formulae I, Ia, Ib, II, IIa, IIb, IIai, IIaii, IIbi, or IIbii, wherein R³ is methyl. In some embodiments, the opioid drug is a mu-opioid agonist according to any of Formulae I, Ia, Ib, II, IIa, IIb, IIai, IIaii, IIbi, or IIbii, wherein R³ is ethyl. In other embodiments, the opioid drug is a mu-opioid agonist according to any of Formulae I, Ia, Ib, II, IIa, IIb, IIai, IIaii, IIbi, or IIbii, wherein R³ is —C(O)CH₃.

In additional embodiments, the opioid drug is a mu-opioid agonist according to any of Formulae I, Ia, Ib, II, IIa, IIb, IIai, IIaii, IIbi, or IIbii, wherein R⁴ is methyl. In some embodiments, the opioid drug is a mu-opioid agonist according to any of Formulae I, Ia, Ib, II, IIa, IIb, IIai, IIaii, IIbi, or IIbii, wherein R⁴ is ethyl. In other embodiments, the opioid drug is a mu-opioid agonist according to any of Formulae I, Ia, Ib, II, IIa, IIb, IIai, IIaii, IIbi, or IIbii, wherein R⁴ is —C(O)CH₃.

In yet further embodiments, the opioid drug is a mu-opioid agonist according to any of Formulae I, Ia, or Ib, wherein R⁵ is —C(O)—. In some embodiments, the opioid drug is a mu-opioid agonist according to any of Formulae I, Ia, or Ib, wherein R⁵ is —CH(OH)—. In some embodiments, the opioid drug is a mu-opioid agonist according to any of Formulae I, Ia, or Ib, wherein R⁵ is —CH(OCH₃)—. In yet other embodiments, the opioid drug is a mu-opioid agonist according to any of Formulae I, Ia, or Ib, wherein R⁵ is —CH(OCH₂CH₃)—. In some embodiments, the opioid drug is a mu-opioid agonist according to any of Formulae I, Ia, or Ib, wherein R⁵ is —CH(OC(O)CH₃)—.

In some embodiments, the opioid drug is a mu-opioid agonist according to any of Formulae I, Ia, or Ib, wherein R⁶ is hydrogen. In other embodiments, the opioid drug is a mu-opioid agonist according to any of Formulae I, Ia, or Ib, wherein R⁶ is methyl. In other embodiments, the opioid drug is a mu-opioid agonist according to any of Formulae I, Ia, or Ib, wherein R⁶ is ethyl. In additional embodiments, the opioid drug is a mu-opioid agonist according to any of Formulae I, Ia, or Ib, wherein R⁶ is —C(O)CH₃.

In some embodiments, the opioid drug is a mu-opioid agonist according to any of Formulae I, Ia, or Ib, wherein E¹ is hydrogen, methyl, or hydroxyl. In an embodiment, the opioid drug is a mu-opioid agonist according to any of Formulae I, Ia, or Ib, wherein E¹ is C1-C10 alkyl. In other embodiments, the opioid drug is a mu-opioid agonist according to any of Formulae I, Ia, or Ib, wherein E¹ is hydrogen. In additional embodiments, the opioid drug is a mu-opioid agonist according to any of Formulae I, Ia, or Ib, wherein E¹ is methyl. In further embodiments, the opioid drug is a mu-opioid agonist according to any of Formulae I, Ia, or Ib, wherein E¹ is hydroxyl.

In one or more embodiments, the opioid drug is a mu-opioid agonist according to any of Formulae I, Ia, Ib, II, IIa, IIb, IIai, IIaii, IIbi, or IIbii, wherein n is a positive integer selected over the range of 1 to 25. For example, in some embodiments, the opioid drug is a mu-opioid agonist according to any of Formulae I, Ia, Ib, II, IIa, IIb, IIai, IIaii, IIbi, or IIbii, wherein n is a positive integer selected over the range of 1 to 20. In other embodiments, the opioid drug is a mu-opioid agonist according to any of Formulae I, Ia, Ib, II, IIa, IIb, IIai, IIaii, IIbi, or IIbii, wherein n is a positive integer selected over the range of 1 to 15. In additional embodiments, the opioid drug is a mu-opioid agonist according to any of Formulae I, Ia, Ib, II, IIa, IIb, IIai, IIaii, IIbi, or IIbii, wherein n is a positive integer selected over the range of 1 to 12. In other embodiments, the opioid drug is a mu-opioid agonist according to any of Formulae I, Ia, Ib, II, IIa, IIb, IIai, IIaii, IIbi, or IIbii wherein n is a positive integer selected over the range of 1 to 10. In further embodiments, the opioid drug is a mu-opioid agonist according to any of Formulae I, Ia, Ib, II, IIa, IIb, IIai, IIbi, or IIbii, wherein n is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30. In some embodiments, the opioid drug is a mu-opioid agonist according to any of Formulae I, Ia, Ib, II, IIa, IIb, IIai, IIaii, IIbi, or IIbii, wherein n is 1, 2, 3, 4, 5, 6, 7, or 8. In other embodiments, the opioid drug is a mu-opioid agonist according to any of Formulae I, Ia, Ib, II, IIa, IIb, IIai, IIaii, IIbi, or IIbii, wherein n is 9, 10, 11, 12, 13, 14, 15, 16, 17, 18, 19, or 20. In additional embodiments, the opioid drug is a mu-opioid agonist according to any of Formulae I, Ia, Ib, II, IIa, IIb, IIai, IIaii, IIbi, or IIbii, wherein n is 21, 22, 23, 24, 25, 26, 27, 28, 29 or 30.

Amount of Opioid Drug

The amount of the opioid drug in a solid dosage form should be a therapeutically acceptable amount. In some embodiments, the weight of the opioid drug is from 15% to 75% of the total weight of a solid dosage form. In other embodiments, the weight of the opioid drug is from 25% to 65% of the total weight of a solid dosage form. In additional embodiments, the weight of the opioid drug is from 27% to 63% of the total weight of a solid dosage form.

In one or more embodiments, the weight of the opioid drug is from about 25% to 75%, about 25% to 65%, about 25% to 50%, about 25% to 35%, or about 25% to 30% of the total weight of a solid dosage form. In other embodiments, the weight of the opioid drug is from 27% to 33% of the total weight of a solid dosage form. In further embodiments, the weight of the opioid drug is from 28% to 32% of the total weight of a solid dosage form.

In some embodiments, the weight of the opioid drug is from 55% to 65% of the total weight of a solid dosage form. In other embodiments, the weight of the opioid drug is from 57% to 63% of the total weight of a solid dosage form. In yet further embodiments, the weight of the opioid drug is from 58% to 62% of the total weight of a solid dosage form.

In some embodiments, the opioid drug includes a covalently attached water-soluble, non-peptidic oligomer, examples of which are provided above. In some embodiments, the oligomer is a poly(ethylene oxide) such as poly(ethylene glycol). In some embodiments, the opioid drug is α-6-mPEGn-O-oxycodol, wherein n is an integer selected from 1 to 30, or 1 to 10, or a pharmaceutically acceptable salt thereof. In some embodiments, the poly(ethylene oxide) may be a methoxy end-capped oligo(ethylene oxide) having a molecular weight of about 75 (n=1), 119, 163, 207, 251, 295, 339, 383, 427, or 471 daltons. In some embodiments, the opioid drug has a molecular weight of about 390-786 g/mol (based on a molecular weight of 315.364 Da for oxycodone and 44 Da for each PEG monomer added to a single (—OCH₂CH₂OCH₃ group).

Molecular weight in the context of a water-soluble polymer, such as PEG, can be expressed as either a number average molecular weight or a weight average molecular weight. Unless otherwise indicated, all references to molecular weight herein refer to the weight average molecular weight. Both molecular weight determinations, number average and weight average, can be measured using gel permeation chromatography or other liquid chromatography techniques (e.g. gel filtration chromatography). Most commonly employed are gel permeation chromatography and gel filtration chromatography. Other methods for determining molecular weight include end group analysis or the measurement of colligative properties (e.g., freezing-point depression, boiling-point elevation, or osmotic pressure) to determine number average molecular weight or the use of light scattering techniques, ultracentrifugation, MALDI TOF, or viscometry to determine weight average molecular weight. PEG polymers are typically polydisperse (i.e., the number average molecular weight and the weight average molecular weight of the polymers are not equal), possessing low polydispersity values of preferably less than about 1.2, more preferably less than about 1.15, still more preferably less than about 1.10, yet still more preferably less than about 1.05, and most preferably less than about 1.03, depending upon the size of the PEG, its method of production and the like.

In some embodiments, a solid dosage form comprises from 50 milligrams to 1000 milligrams of an opioid drug. In other embodiments, a solid dosage form comprises from 50 milligrams to 900 milligrams of an opioid drug. In yet other embodiments, a solid dosage form comprises from 100 milligrams to 900 milligrams of an opioid drug. In other embodiments, a solid dosage form comprises from 100 milligrams to 800 milligrams of an opioid drug. In some embodiments, a solid dosage form comprises from 50 milligrams to 150 milligrams of an opioid drug. In other embodiments, a solid dosage form comprises from 75 milligrams to 125 milligrams of an opioid drug. In some embodiments, a solid dosage form comprises from 150 milligrams to 250 milligrams of an opioid drug. In other embodiments, a solid dosage form comprises from 175 milligrams to 225 milligrams of an opioid drug. In some embodiments, a solid dosage form comprises from 250 milligrams to 350 milligrams of an opioid drug. In other embodiments, a solid dosage form comprises from 275 milligrams to 325 milligrams of an opioid drug. In additional embodiments, a solid dosage form comprises from 350 milligrams to 450 milligrams of an opioid drug. In yet other embodiments, a solid dosage form comprises from 375 milligrams to 425 milligrams of an opioid drug. In some other embodiments, a solid dosage form comprises from 450 milligrams to 550 milligrams of an opioid drug. In further embodiments, a solid dosage form comprises from 475 milligrams to 525 milligrams of an opioid drug. In some additional embodiments, a solid dosage form comprises from 550 milligrams to 650 milligrams of an opioid drug. In some embodiments, a solid dosage form comprises from 575 milligrams to 625 milligrams of an opioid drug. In other embodiments, a solid dosage form comprises from 650 milligrams to 750 milligrams of an opioid drug. In additional embodiments, a solid dosage form comprises from 675 milligrams to 725 milligrams of an opioid drug. In other embodiments, a solid dosage form comprises from 750 milligrams to 850 milligrams of an opioid drug. In some embodiments, a solid dosage form comprises from 775 milligrams to 825 milligrams of an opioid drug. In an additional embodiments, a solid dosage form comprises from 850 milligrams to 950 milligrams of an opioid drug. In other embodiments, a solid dosage form comprises from 875 milligrams to 925 milligrams of an opioid drug.

Pharmaceutically Acceptable Salts

The opioid drugs described herein include not only the opioid drugs themselves, but the opioid drugs in the form of a pharmaceutically acceptable salt as well. An opioid drug as described herein can possess a sufficiently acidic group, a sufficiently basic group, or both functional groups, and, accordingly, react with any of a number of inorganic bases, and inorganic and organic acids, to form a salt.

Acids for forming acid addition salts are known to those of skill in the art and include hydrochloric acid, hydrobromic acid, hydroiodic acid, sulfuric acid, phosphoric acid, p-toluenesulfonic acid, methanesulfonic acid, oxalic acid, p-bromophenyl-sulfonic acid, carbonic acid, succinic acid, citric acid, benzoic acid, and acetic acid, among others. Examples of such pharmaceutically acceptable salts include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, gamma hydroxybutyrate, glycolate, tartrate, methanesulfonate, propanesulfonate, naphthalene 1-sulfonate, naphthalene-2-sulfonate, and mandelate salts, among others.

Bases for forming base addition salts are known to those of skill in the art and include ammonium hydroxides, alkali hydroxides, alkaline earth metal hydroxides, carbonates, and bicarbonates, among others. Specific bases for forming base addition salts are known to those of skill in the art and include sodium hydroxide, potassium hydroxide, ammonium hydroxide, and potassium carbonate, among others.

Other Pharmaceutically Acceptable Excipients

In addition to an opioid drug and a high viscosity agent, a solid dosage form can contain one or more inactive, pharmaceutically acceptable carrier materials (excipients) such as, without limitation, binders, lubricants, disintegrants, fillers, stabilizers, surfactants, carbohydrates, inorganic salts, antimicrobial agents, antioxidants, buffers, acids, bases, coloring agents, and the like. Excipients may be provided as an intra-granular, extra-granular, or both an intra-granular and extra-granular component of a solid dosage form.

For example, in some embodiments, the solid dosage form comprises one or more binders. Binders can impart cohesive qualities to a solid dosage form, and thus ensure that the solid dosage form remains intact. Suitable binder materials include, but are not limited to, starch (including corn starch and pregelatinized starch), gelatin, sugars (including sucrose, glucose, dextrose and lactose), polyethylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, microcrystalline cellulose, ethyl cellulose, hydroxyethyl cellulose, and the like), and magnesium aluminum silicate (e.g., VEEGUM® available from Vanderbildt Minerals, LLC).

The solid dosage form may also comprise one or more lubricants. Lubricants can facilitate manufacture of a solid dosage form by promoting powder flow and/or preventing particle capping (i.e., particle breakage) when pressure is relieved. Useful lubricants include, but are not limited to, magnesium stearate, calcium stearate, and stearic acid.

The solid dosage form may comprise one or more disintegrants. Disintegrants can facilitate disintegration of a solid dosage form, and include, but are not limited to, starches, clays, celluloses, algins, gums, and crosslinked polymers.

In some embodiments, the solid dosage form comprises one or more fillers. Fillers include, for example, materials such as silicon dioxide, titanium dioxide, alumina, talc, kaolin, powdered cellulose, and microcrystalline cellulose, as well as soluble materials such as mannitol, urea, sucrose, lactose, dextrose, sodium chloride, and sorbitol.

In some embodiments, the solid dosage form comprises one or more stabilizers. Stabilizers can inhibit or retard drug decomposition reactions that include, by way of example, oxidative reactions.

In some embodiments, the solid dosage form comprises one or more carbohydrates. A carbohydrate such as a sugar, a derivatized sugar such as an alditol, aldonic acid, an esterified sugar, and/or a sugar polymer can be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, sorbitol (glucitol), pyranosyl sorbitol, myoinositol, and the like.

In some embodiments, the solid dosage form comprises one or more inorganic salts or buffers. The excipient can also include, without limitation, an inorganic salt or buffer such as citric acid, sodium chloride, potassium chloride, sodium sulfate, potassium nitrate, sodium phosphate monobasic, sodium phosphate dibasic, and combinations thereof.

In some embodiments, the solid dosage includes one or more antimicrobial agents for preventing or deterring microbial growth. Non-limiting examples of antimicrobial agents suitable for a solid dosage form include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

In some embodiments, the solid dosage form comprises one or more antioxidants. Antioxidants can prevent oxidation, thereby preventing deterioration of components of a solid dosage form. Suitable antioxidants for use in a solid dosage form include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

In some embodiments, the solid dosage form comprises one or more surfactants.

Exemplary surfactants include without limitation: polysorbates, such as TWEEN® 20 and TWEEN® 80,” and polyoxyalkylene ethers such as PLURONIC® F68 and F88 (both of which are available from BASF, Mount Olive, N.J.); sorbitan esters; lipids, including phospholipids such as lecithin and other phosphatidylcholines, phosphatidylethanolamines (although preferably not in liposomal form), fatty acids and fatty esters; steroids, such as cholesterol; and chelating agents, such as ethylenediaminetetraacetic acid (EDTA), zinc and other such suitable cations.

The compositions, formulations and solid dosage forms can take any number of forms and are not limited in this regard. Preferred exemplary preparations are suitable for oral delivery. In some embodiments, the solid dosage forms may be any one of a tablet, caplet, capsule, gel cap, troche, dispersion, lozenge, suppository, granules, beads, pellets or a powder. Such dosage forms are prepared using conventional methods known to those in the field of pharmaceutical formulation and as described in the pertinent texts. Exemplary methods of forming tablets are provided in the examples herein.

In some embodiments, the solid dosage form comprises one or more acids or bases. Non-limiting examples of acids that can be used include hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

In some embodiments, the solid dosage form comprises one or more of polyethylene glycol (PEG) (e.g., Mol. Wt. 3350), polyethylene oxide (PEO, e.g., Mol. Wt. 400K, 4,000,000, or 8,000,000) (e.g., POLYOX™ WSR 308 (Mol. Wt. 8 million), POLYOX™ WSR 303 (Mol. Wt. 7 million), POLYOX™ WSR 301 (Mol. Wt. 4 million), POLYOX™ WSR Coagulant (Mol. Wt. 5 million), POLYOX™ WSR 1105 (Mol. Wt. 900,000)), sodium alginate (e.g., PROTANAL® PH 6160), xanthan gum (e.g., XANTURAL® 75, XANTURAL® 180), hydroxypropylcellulose (e.g., KLUCEL™ JXF Pharm, KLUCEL™ MF, KLUCEL™ GXF), hydroxypropylmethylcellulose (e.g., K100M), carrageenan (e.g., GELCARIN® GP 379), poly acrylic acid (PAA) (e.g., Carbomer 943 p, CARBOPOL® 934), cetostearyl alcohol, agar, sodium carboxymethylcellulose (NaCMC) (e.g. AQUALON® CMC 7HF or 7HXF), ethyl cellulose, acacia, carnuaba wax, or combinations thereof.

In certain embodiments, an excipient in a solid dosage form is microcrystalline cellulose (Avicel PH 101), dibasic calcium phosphate anhydrous (Fujicalin), xanthan gum, croscarmellose sodium (AC-DI-SOL®), colloidal silicon dioxide, polyvinylpyrrolidone (PVP) (e.g. Povidone K29-32), NaCMC, microcrystalline cellulose (Avicel PH 102), colloidal silicon dioxide, magnesium stearate, purified water, or a combination thereof.

In some additional embodiments, an excipient in a solid dosage form is selected from microcrystalline cellulose (Avicel PH 101), dibasic calcium phosphate anhydrous (Fujicalin), croscarmellose sodium (AC-DI-SOL®), colloidal silicon dioxide, Povidone K29-32, purified water, xanthan gum, NaCMC, polyethylene oxide, microcrystalline cellulose (Avicel PH 102), magnesium stearate, or a combination thereof.

In other embodiments, an excipient in a solid dosage form is selected from microcrystalline cellulose (Avicel PH 101), dibasic calcium phosphate anhydrous (Fujicalin), xanthan gum (XANTURAL® 180), croscarmellose sodium (AC-DI-SOL®), colloidal silicon dioxide (CAB-O-SIL® M5P), Povidone (PLASDONE™ K-29/32), purified water, sodium carboxymethyl cellulose (AQUALON® 7HXF or 7HF), microcrystalline cellulose (Avicel PH 102), magnesium stearate, or a combinations thereof.

An exemplary formulation comprises α-6-mPEGₙ-O-oxycodol phosphate (where n is an integer selected from 1-10) (100-250 mg/tablet), microcrystalline cellulose (200-350 mg/tablet), dibasic calcium phosphate anhydrous (90-250 mg/tablet), croscarmellose sodium (5-50 mg/tablet), colloidal silicon dioxide (5-50 mg/tablet), a polyvinylpyrrolidone such as a Povidone (5-20 mg/tablet), magnesium stearate (5-10 mg/tablet), and the high viscosity agent.

Methods of Administration

In certain embodiments, provided herein are methods for administering a solid dosage form described herein. In certain embodiments, the method comprises administering a composition as provided herein to a patient suffering from a condition that is responsive to treatment with an opioid agonist. In certain embodiments, the method comprises administering a solid dosage form described herein. The method of administering may be used to treat any condition that can be remedied or prevented by administration of an opioid drug (e.g., moderate to severe pain). In some preferred embodiments, the composition is used for treating moderate to severe chronic low back pain. As the cause of the pain is not necessarily critical to the methods disclosed herein, the methods include the treatment of pain arising from various sources, injuries, and disease states. Those of ordinary skill in the art appreciate which conditions an opioid drug can effectively treat, for example, nociceptive pain. In certain embodiments, the condition includes neuropathic pain. The actual dose administrated will vary depending on the age, weight, and general condition of the subject as well as the severity of the condition being treated, the judgment of the health care professional, and the active ingredient being administered. Therapeutically effective amounts are described herein.

The solid dosage forms described herein can be administered in a variety of dosing schedules depending on the judgment of the clinician, needs of the patient, and so forth. The specific dosing schedule will be known by those of ordinary skill in the art or can be determined experimentally using routine methods. Exemplary dosing schedules include, without limitation, administration five times a day, four times a day, three times a day, twice daily, once daily, three times weekly, twice weekly, once weekly, twice monthly, once monthly, and any combination thereof. In certain embodiments, a solid dosage form is administered as necessary over a 24 hour period to manage moderate to severe pain. Management of moderate to severe pain includes treating and/or preventing pain. In certain embodiments, a solid dosage form is administered as necessary over a 24 hour period to treat and/or prevent moderate to severe pain. In certain embodiments, a solid dosage form is administered as necessary over a 24 hour period to treat moderate to severe pain. In certain embodiments, a solid dosage form is administered as necessary over a 24 hour period to prevent moderate to severe pain.

In yet another particular embodiment, a solid dosage form is provided for use in the manufacture of a medicament for the treatment of pain.

### EXAMPLES

It is to be understood that while certain preferred and specific embodiments have been described, the foregoing description as well as the examples that follow are intended to illustrate and not limit the scope of the claims. Other aspects, advantages and modifications within the scope of the claims will be apparent to those skilled in the relevant art.

The following materials were used in for the formulations in the Examples, unless otherwise noted.

| Name | Supplier |
|---|---|
| Polyethylene Glycol (PEG) 3350, NF | Spectrum |
| Polyethylene Oxide (PEO), NF (Mol. Wt. 400K) | Sigma-Aldrich |
| POLYOX ™ WSR 308 NF Grade (Mol. Wt. 8 million) | Dow |
| POLYOX ™ WSR 303 NF Grade (Mol. Wt. 7 million) | Dow |
| POLYOX ™ WSR 301 NF Grade (Mol. Wt: 4 million) | Dow |
| POLYOX ™ WSR Coagulant NF Grade (Mol. Wt: 5 million) | Union Carbide Corp. |
| POLYOX ™ WSR 1105 NF Grade (Mol. Wt: 900,000) | Dow |
| PROTANAL ® PH 6160 (Xanthan gum), USP | FMC Biopolymer |
| XANTURAL ® 75 (Sodium Alginate), USP | CPKELCO |
| XANTURAL ® 180 (Sodium Alginate), USP | CPKELCO |
| KLUCEL ™ DCF Pharm (Hydroxypropylcellulose), NF | Ashland |
| KLUCEL ™ NIF (Hydroxypropylcellulose), NF | Ashland |
| KLUCEL ™ GXF (Hydroxypropylcellulose), NF | Ashland |
| Hydroxy propyl methylcellulose (HPMC) K100M | Colorcon |
| GELCARIN ® GP 379 (Carrageenan), NF | FMC Biopolymer |
| Carbomer 943 p, NF | Spectrum |
| Cetostearyl alcohol, NF | Spectrum |
| Agar, NF | Spectrum |
| Carboxymethyl Cellulose Sodium (NaCMC) | Spectrum |
| High Viscosity, Viscosity of 1 Percent Aqueous Solution | |
| at 25 DEG C.: 1500-3000 cP, USP (CAS no. 9004-32-4) | |
| Ethyl cellulose, NF | Spectrum |
| Acacia, NF | Spectrum |
| Carnuaba Wax | Spectrum |
| CARBOPOL ® 934, NF | Spectrum/Lubrizol |
| PEO (Mol. Wt: 8,000,000), NF | Spectrum/Dow |
| PEO (Mol. Wt: 900,000), NF | Spectrum/Dow |
| PEO (Mol. Wt: 4,000,000), NF | Spectrum/Dow |
| Carboxymethyl cellulose Sodium (NaCMC) | Ashland |
| AQUALON ® polymer, 7HXF or 7HF | |

### Example 1

### Oral Tablet Formulations without Active Agent and Initial Testing

Oral tablet formulations without active agent for initial testing were prepared and tested according to the following methodology.

The weighed quantity of the polymers/combination of polymers was transferred to a Sieve #20. The sieved materials were collected and mixed for about 5 minutes to achieve visible homogeneity. About one tablet equivalent (150-300 mg) was weighed and compressed using a Ø7.1 mm round, standard concave, B tool.

The tablets/compacts were evaluated for hardness, tablet weight gain after hydration/swellability, and granules blend hydration/swellability after 3 hours. The granules blends were evaluated for gelling capacity in both purified water and/or ethanol (40% or 200% proof) over a period of time. Swellability and hydration was carried out by taking initial weight of the tablet, addition of 1.0/1.5 ml of water or ethanol in a scintillation vial containing the tablet, and weighing of the hydrated tablet after 3 hours for calculation of percent hydration/swellability. Blend hydration and gelling of powder blend equivalent to the tablet weight was carried out by taking initial weight of the tablet, adding 1.0/1.5 ml of water or ethanol in a scintillation vial containing the tablet, and weighing the hydrated tablet after 3 hours for calculation of percent hydration/swellability.

Initial formulations and initial testing results are provided below.

| | | Evaluations | | | | | |
|---|---|---|---|---|---|---|---|
| | | | | TABLET | | BLEND | |
| | | | INITIAL | WT. GAIN | | HYDRATION | |
| | | | TABLET | (MG) | % TABLET | AFTER 3 HRS. | |
| | | HARD-NESS | WEIGH | AFTER 3 | WEIGHT | GEL | |
| # | Composition | (KP) | T (MG) | HRS. | GAIN | FORMATION | REMARKS |
| 1 | PEG 3350 + | 22-24 | 300 | 698 | 132.67 | ✓ | NA |
| | PEO | | | | | | |
| | (Mol. Wt: | | | | | | |
| | 400,000), 1:2 | | | | | | |
| | Ratio | | | | | | |
| 2 | PEG 3350 + | 9-10 | 150 | 503 | 235.33 | ✓ | NA |
| | POLYOX™ | | | | | | |
| | WSR 308 | | | | | | |
| | (Mol. Wt: | | | | | | |
| | 8,000,000), | | | | | | |
| | 1:2 Ratio | | | | | | |
| 3 | PEG 3350 + | 9-10 | 150 | 479 | 219.33 | ✓ | NA |
| | POLYOX™ | | | | | | |
| | WSR 303 | | | | | | |
| | (Mol. Wt: | | | | | | |
| | 7,000,000), | | | | | | |
| | 1:2 Ratio | | | | | | |
| 4 | PEG 3350 + | 9-10 | 150 | 430 | 186.67 | ✓ | NA |
| | POLYOX™ | | | | | | |
| | WSR 301 | | | | | | |
| | (Mol. Wt: | | | | | | |
| | 4,000,000), | | | | | | |
| | 1:2 Ratio | | | | | | |
| 5 | PEG 3350 + | 9-10 | 150 | 438 | 192.00 | ✓ | NA |
| | POLYOX™ | | | | | | |
| | WSR | | | | | | |
| | Coagulant | | | | | | |
| | (Mol. Wt: | | | | | | |
| | 5,000,000), | | | | | | |
| | 1:2 Ratio | | | | | | |
| 6 | PEG 3350 + | 9-10 | 150 | 378 | 152.00 | ✓ | NA |
| | POLYOX™ | | | | | | |
| | WSR 1105 | | | | | | |
| | (Mol. Wt: | | | | | | |
| | 900,000), | | | | | | |
| | 1:2 | | | | | | |
| | Ratio | | | | | | |
| 7 | PEG 3350 + | 11-13 | 150 | 393 | 162.00 | ✓ | Electrostatic and |
| | HPMC | | | | | | does not have |
| | K100 M, 1:2 | | | | | | good wetting of |
| | Ratio | | | | | | the blend |
| 8 | PEG 3350 + | 4-5 | 150 | 660 | 340.00 | ✓ | NA |
| | PROTANAL | | | | | | |
| | ® PH 6160, | | | | | | |
| | 1:2 Ratio | | | | | | |
| 9 | PEG 3350 + | 10-11 | 150 | 653 | 335.33 | ✓ | Tablets have rough |
| | XANTURA | | | | | | surface |
| | L® 75, 1:2 | | | | | | |
| | Ratio | | | | | | |
| 10 | PEG 3350 + | 5-6 | 150 | 607 | 304.67 | ✓ | Tablets have rough |
| | XANTURA | | | | | | surface |
| | L® 180, 1:2 | | | | | | |
| | Ratio | | | | | | |
| 11 | PEG 3350 + | 14-15 | 150 | 190 | 26.67 | X | Electrostatic, |
| | KLUCEL™ | | | | | | fluffy material, |
| | JXF, 1:2 | | | | | | tablets stick to the |
| | Ratio | | | | | | upper punch and |
| | | | | | | | does not have |
| | | | | | | | good wetting of |
| | | | | | | | the blend |
| 12 | PEG 3350 + | 12-13 | 150 | 235 | 56.67 | X | Electrostatic, |
| | KLUCEL™ | | | | | | fluffy material, |
| | GXF, 1:2 | | | | | | tablets stick to the |
| | Ratio | | | | | | upper punch and |
| | | | | | | | doesn’t have |
| | | | | | | | wetting of the |
| | | | | | | | blend |
| 21 | PEG 3350 + | NT | | | | | Fluffy, static and |
| | CARBOPOL | | | | | | couldn't compress |
| | ® 934 p, 1:2 | | | | | | the tablets |
| | Ratio | | | | | | |
| 22 | PEG 3350 + | 8-9 | 150 | 781 | 420.67 | ✓ | Good hydration |
| | GELCARIN | | | | | | and less soluble in |
| | ® GP 379, | | | | | | alcohol |
| | 1:2 Ratio | | | | | | |
| 23 | PEG 3350 + | 3-4 | 150 | 150 | 0.00 | X | Difficult to |
| | Cetostrearyl | | | | | | compress the |
| | alcohol, 1:2 | | | | | | tablets as |
| | Ratio | | | | | | cetostearyl alcohol |
| | | | | | | | has no |
| | | | | | | | compressibility |
| 24 | PEG 3350 + | 7-8 | 150 | 488 | 225.33 | ✓ | Tablet is intact in |
| | PEO | | | | | | alcohol. |
| | (Mol.Wt: | | | | | | |
| | 8,000,000) + | | | | | | |
| | PEO | | | | | | |
| | (Mol.Wt: | | | | | | |
| | 400,000) | | | | | | |
| 25 | PEG 3350 + | 7-8 | 150 | 615 | 310.00 | ✓ | Tablet is intact in |
| | PEO | | | | | | alcohol. |
| | (Mol.Wt: | | | | | | |
| | 8,000,000) + | | | | | | |
| | XANTURA | | | | | | |
| | L® 180 | | | | | | |
| | (xanthan | | | | | | |
| | gum) | | | | | | |
| 26 | PEG 3350 + | 7-8 | 150 | 595 | 296.67 | ✓ | Tablet is intact in |
| | PEO | | | | | | alcohol. |
| | (Mol.Wt: | | | | | | |
| | 8,000,000)+ | | | | | | |
| | ® PH 6160 | | | | | | |
| | (sodium | | | | | | |
| | alginate) | | | | | | |
| 30 | PEG 3350 + | 6-7 | 150 | NT | | X | Good |
| | Agar, 1:2 | | | | | | compressibility. |
| | Ratio | | | | | | No sticking to the |
| | | | | | | | punches and dies. |
| | | | | | | | Completely |
| | | | | | | | dissolved in water |
| | | | | | | | and alcohol after 3 |
| | | | | | | | hrs of hydration. |
| 31 | PEG 3350 + | 7-8 | 150 | 914 | 509.33 | ✓ | Good |
| | Na CMC, 1:2 | | | | | | compressibility. |
| | Ratio | | | | | | No sticking to the |
| | | | | | | | punches and dies. |
| | | | | | | | Tablet is intact in |
| | | | | | | | alcohol |
| 32 | PEG 3350 + | 6-7 | 150 | NT | | X | Good |
| | Ethyl | | | | | | compressibility. |
| | Cellulose, | | | | | | No sticking to the |
| | 1:2 Ratio | | | | | | punches and dies. |
| | | | | | | | No swellability in |
| | | | | | | | water and little |
| | | | | | | | swellability in |
| | | | | | | | alcohol. |
| 33 | PEG 3350 + | 7-8 | 150 | NT | | X | Good |
| | Acacia gum, | | | | | | compressibility. |
| | 1:2 Ratio | | | | | | No sticking to the |
| | | | | | | | punches and dies. |
| | | | | | | | Completely |
| | | | | | | | dissolved in water |
| | | | | | | | and alcohol after 3 |
| | | | | | | | hrs of hydration. |
| 34 | PEG 3350 + | 4-5 | 150 | NT | | X | Compressibility- |
| | Carnuaba | | | | | | Sticking to both |
| | Wax, 1:2 | | | | | | lower and upper |
| | Ratio | | | | | | punches. In |
| | | | | | | | alcohol dispersion |
| | | | | | | | formed after 3 hrs. |
| 35 | PEG 3350 + | 4-5 | 150 | NT | | X | Compressibility- |
| | Cetostearyl | | | | | | Sticking to both |
| | alcohol, 1:2 | | | | | | lower and upper |
| | Ratio | | | | | | punches. In |
| | | | | | | | alcohol dispersion |
| | | | | | | | formed after 3 hrs. |
| 42 | Solid | 4-5 | 150 | NT | | | Good |
| | Dispersion | | | | | | compressibility. |
| | PEG 3350 + | | | | | | No sticking to the |
| | Cetostearyl | | | | | | punches and dies |
| | alcohol (1:1) | | | | | | |

NT = Not Tested
X = Viscous gel was not observed
✓ = Viscous gel was observed

As seen in the tables above, compositions comprising a number of polymers formed a gel after 3 hours when placed in water or ethanol. Further, compositions comprising a number of polymers provided a high swelling rate (e.g. at least 200% tablet weight gain in solution) when hydrated for three hours. Tablets comprising PEG 3350 and NaCMC (formulation #31) had a tablet weight gain of over 500%.

### Example 2

### Oral Tablet Formulations with Active Agent and Initial Testing

Oral tablet formulations with active agent for initial testing were prepared and tested according to the following methodology.

Twenty (21) active agent formulations were prepared with the selected polymer or a combination of polymers. The strength of each formulation was 50 mg. Batch size of each formulation trial was 4-10 g.

Formulations 13-20: α-6-mPEG₆-O-oxycodol phosphate and selected polymers, either alone or in combination were weighed or mixed mechanically for 5 minutes. Blend percentage ratio of α-6-mPEG₆-O-oxycodol phosphate and polymer were 29.07:70.93.

Formulations 37-40: α-6-mPEG₆-O-oxycodol phosphate and combination of polymers were blended at percentage ratio of 29.07:56.75:14.18.

Formulation 41: α-6-mPEG₆-O-oxycodol phosphate and cetostearyl alcohol were melted at 750° C. Homogeneous paste formed. The paste was cooled at room temperature, passed through a mesh, and compressed into tablets/compacts.

Formulations 43-46: Coated tablets prepared from α-6-mPEG₆-O-oxycodol phosphate were used. Twenty five (25) coated tablets were crushed to powder using a mortar and pestle and passed through a mesh.

In general, the blending and compression was carried out as follows. Required quantities of crushed powder and selected polymer (10% of tablet weight) were weighed and then mixed. The prepared blend was then compressed. The tablets/compacts were evaluated for hardness, tablet weight gain after hydration/swellability, and the granules blend hydration/swellability after 3 hours. The granules blend was evaluated for gelling capacity in both purified water and/or ethanol (40% or 200% proof) over a period of time. Determinations of swellability and hydration were carried out by measuring the initial weight of the tablet, addition of 1.0/1.5 ml of water or ethanol in a scintillation vial containing the tablet, and weighing of the hydrated tablet after 3 hours for calculation of percent hydration/swellability.

Initial formulations with active agent and initial testing results are provided below, where the active pharmaceutical ingredient (API) is α-6-mPEG₆-O-oxycodol phosphate.

| | | Evaluations | | | | |
|---|---|---|---|---|---|---|
| | | | | TABLET | | BLEND |
| | | | INITIAL | WT. GAIN | | HYDRATION |
| | | | TABLET | (MG) | % TABLET | AFTER 3 HRS. |
| | | HARDNESS | WEIGH | AFTER 3 | WEIGHT | GEL |
| # | Composition | (KP) | T (MG) | HRS. | GAIN | FORMATION |
| 13 | API, 50 mg | 8-9 | 200 | 491 | 145.50 | ✓ |
| | (Base | | | | | |
| | Equivalent) | | | | | |
| | + PEO | | | | | |
| | (Mol. Wt: | | | | | |
| | 400,000) | | | | | |
| 14 | API, 50 mg | 7-8 | 200 | 543 | 171.50 | ✓ |
| | (Base | | | | | |
| | Equivalent) | | | | | |
| | + | | | | | |
| | POLYOX™ | | | | | |
| | WSR 1105 | | | | | |
| | NF Grade | | | | | |
| | (Mol. Wt: | | | | | |
| | 900,000 | | | | | |
| 15 | API, 50 mg | 8-9 | 200 | 627 | 213.50 | ✓ |
| | (Base | | | | | |
| | Equivalent) | | | | | |
| | + | | | | | |
| | POLYOX™ | | | | | |
| | WSR 301 NF | | | | | |
| | Grade | | | | | |
| | (Mol.Wt: | | | | | |
| | 4,000,000) | | | | | |
| 16 | API, 50 mg | 7-8 | 200 | 674 | 237.00 | ✓ |
| | (Base | | | | | |
| | Equivalent) | | | | | |
| | + | | | | | |
| | POLYOX™ | | | | | |
| | WSR 308 NF | | | | | |
| | Grade | | | | | |
| | (Mol.Wt: | | | | | |
| | 8,000,000) | | | | | |
| 17 | API, 50 mg | 8-9 | 200 | 542 | 171.00 | ✓ |
| | (Base | | | | | |
| | Equivalent) | | | | | |
| | + HPMC | | | | | |
| | K100 M | | | | | |
| 18 | API, 50 mg | 7-8 | 200 | 859 | 329.50 | ✓ |
| | (Base | | | | | |
| | Equivalent) | | | | | |
| | + | | | | | |
| | PROTANAL | | | | | |
| | ® PH 6160 | | | | | |
| | (sodium | | | | | |
| | alginate) | | | | | |
| 19 | API, 50 mg | 8-9 | 200 | 857 | 328.50 | X |
| | (Base | | | | | |
| | Equivalent) | | | | | |
| | + | | | | | |
| | XANTURA | | | | | |
| | L® 180 | | | | | |
| | (xanthan | | | | | |
| | gum) | | | | | |
| 20 | API, 50 mg | 8-9 | 200 | 788 | 294.00 | X |
| | (Base | | | | | |
| | Equivalent) | | | | | |
| | + | | | | | |
| | XANTURA | | | | | |
| | L® 75 | | | | | |
| | (xanthan | | | | | |
| | gum) | | | | | |

NA = Not Applicable
X = Viscous gel was not observed
✓ = Viscous gel was observed

| | | Evaluations | | |
|---|---|---|---|---|
| | | | Blend | |
| | | Hardness | hydration | |
| Formulation | Composition | (kp) | after 3 hrs | Remarks |
| 37 | API, 50 mg (Base Equivalent) + | 11-12 | NT | Good flow, |
| | PEO (Mol. Wt: 900,000) + PEO | | | compressibility. No |
| | (Mol. Wt: 400,000) | | | sticking to the punches |
| | | | | and dies. |
| 38 | API, 50 mg (Base Equivalent) + | 9-10 | NT | Good flow, |
| | PEO (Mol. Wt: 900,000) + | | | compressibility. No |
| | XANTURAL ® 180 | | | sticking to the punches |
| | (xanthan gum) | | | and dies. |
| 39 | API, 50 mg (Base Equivalent) + | 9-10 | NT | Good flow, |
| | PEO (Mol. Wt: 900,000) + | | | compressibility. No |
| | PROTANAL ® PH 6160 | | | sticking to the punches |
| | (sodium alginate) | | | and dies. |
| 40 | API, 50 mg (Base Equivalent) + | 9-10 | NT | Good flow, |
| | PEO (Mol. Wt: 900,000) + | | | compressibility. No |
| | GELCARIN ® GP379 | | | sticking to the punches |
| | (carrageenan) | | | and dies. |
| 41 | API, 50 mg (Base Equivalent) + | 4-5 | NT | Compressibility- |
| | Cetostearyl alcohol (Solid | | | Sticking to both lower |
| | Dispersion) | | | and upper punches. |
| 43 | API crushed Tablet powder + | 11-12 | √ | Good compressibility. |
| | PEO (Mol. Wt: 900,000) | | | No sticking to the |
| | | | | punches and dies. |
| 44 | API crushed Tablet powder + | 11-12 | √ | Good compressibility. |
| | Na CMC | | | No sticking to the |
| | | | | punches and dies. |
| 45 | API crushed Tablet powder + | 11-12 | √ | Good compressibility. |
| | XANTURAL ® 180 | | | No sticking to the |
| | (xanthan gum) | | | punches and dies. |
| 46 | API crushed Tablet powder + | 11-12 | √ | Good compressibility. |
| | GELCARIN ® GP 379 | | | No sticking to the |
| | (carrageenan) | | | punches and dies. |

NT = Not Tested
X = Viscous gel was not observed
√ = Viscous gel was observed

As seen above, tablets comprising an active agent and a number of polymers formed a gel after 3 hours when placed in water or ethanol.

The tablets/compacts and powder blends were evaluated for extraction of α-6-mPEG₆-O-oxycodol phosphate in 3-5 mL of purified water and/or ethanol (200% and 40% proof), results are provided below, where API is α-6-mPEG₆-O-oxycodol phosphate.

| | | | | Media | | | | | | |
|---|---|---|---|---|---|---|---|---|---|---|
| | | API | Dissolution | Volume | Percent Dissolved (Average ± SD) | | | | | |
| # | Compact | (mg) | Medium | (mL) | 15 min | 60 min | 120 min | 240 min | | |
| Extraction API Tablets in Water (Formulations 13-20) | | | | | | | | | | |
| 13 | API + | 50 | Water at | 5 | 9.49 ± 0.31 | 17.91 ± 1.03 | 27.64 ± 2.70 | 46.57 ± 4.35 | | |
| | PEO | | RT | | | | | | | |
| | (400 K) | | | | | | | | | |
| 14 | API + | 50 | Water at | 5 | 8.0 ± 0.99 | 17.9 ± 3.02 | 28.7 ± 5.60 | 48.2 ± 8.91 | | |
| | PEO | | RT | | | | | | | |
| | (900 K) | | | | | | | | | |
| 15 | API + | 50 | Water at | 5 | 8.0 ± 0.91 | 16.3 ± 2.17 | 25.3 ± 3.82 | 41.6 ± 6.27 | | |
| | PEO (4 | | RT | | | | | | | |
| | million) | | | | | | | | | |
| 16 | API + | 50 | Water at | 5 | 10.4 ± 0.70 | 21.6 ± 0.77 | 31.0 ± 0.54 | 46.9 ± 0.57 | | |
| | PEO (8 | | RT | | | | | | | |
| | million) | | | | | | | | | |
| 17 | API + | 50 | Water at | 5 | 9.4 ± 0.39 | 19.7 ± 1.59 | 30.7 ± 2.61 | 49.0 ± 2.65 | | |
| | HPMC | | RT | | | | | | | |
| | Kl00 M | | | | | | | | | |
| 18 | API + | 50 | Water at | 5 | 0.6 ± 0.21 | 1.50 ± 0.32 | 3.1 ± 0.53 | 6.1 ± 1.11 | | |
| | PROTA | | RT | | | | | | | |
| | NAL® | | | | | | | | | |
| | PH 6160 | | | | | | | | | |
| | (sodium | | | | | | | | | |
| | alginate) | | | | | | | | | |
| 19 | API + | 50 | Water at | 5 | 1.2 ± 0.21 | 2.5 ± 0.62 | 4.2 ± 0.98 | 8.0 ± 1.68 | | |
| | XANTU | | RT | | | | | | | |
| | RAL® | | | | | | | | | |
| | 180 | | | | | | | | | |
| | (xanthan | | | | | | | | | |
| | gum) | | | | | | | | | |
| 20 | API + | 50 | Water at | 5 | 1.0 ± 0.11 | 2.2 ± 0.07 | 3.8 ± 0.04 | 7.6 ± 0.17 | | |
| | XANTU | | RT | | | | | | | |
| | RAL® | | | | | | | | | |
| | 75 | | | | | | | | | |
| | (xanthan | | | | | | | | | |
| | gum) | | | | | | | | | |
| | | | | Media | | | | | | |
| | | API | Dissolution | Volume | Percent Dissolved (Average ± SD) | | | | | |
| # | Compact | (mg) | Medium | (mL) | 15 min | 60 min | 120 min | 240 min | | |
| Extraction of API Tablets in Ethanol (Formulations 14-20) | | | | | | | | | | |
| 14 | API + | 50 | Ethanol | 3 | 11.61 ± 0.49 | 37.67 ± 2.69 | 53.39 ± 5.07 | 72.55 ± 5.25 | | |
| | PEO | | (200% | | | | | | | |
| | (900 K) | | Proof) | | | | | | | |
| 16 | API + | 50 | Ethanol | 3 | 11.4 ± 0.50 | 26.1 ± 1.41 | 42.1 ± 1.75 | 59.2 ± 4.81 | | |
| | PEO (8 | | (200% | | | | | | | |
| | million) | | Proof) | | | | | | | |
| 17 | API + | 50 | Ethanol | 3 | 10.3 ± 2.35 | 51.1 ± 5.71 | 66.5 ± 4.08 | 74.7 ± 3.60 | | |
| | HPMC | | (200% | | | | | | | |
| | Kl00 M | | Proof) | | | | | | | |
| 18 | API + | 50 | Ethanol | 3 | 7.0 ± 0.26 | 28.3 ± 1.96 | 62.0 ± 0.88 | 78.4 ± 1.89 | | |
| | PROTA | | (200% | | | | | | | |
| | NAL® | | Proof) | | | | | | | |
| | PH 6160 | | | | | | | | | |
| | (Sodium | | | | | | | | | |
| | Alginate) | | | | | | | | | |
| 19 | API + | 50 | Ethanol | 3 | 1.2 ± 0.21 | 2.5 ± 0.62 | 4.2 ± 0.98 | 8.0 ± 1.68 | | |
| | XANTU | | (200% | | | | | | | |
| | RAL® | | Proof) | | | | | | | |
| | 180 | | | | | | | | | |
| | (xanthan | | | | | | | | | |
| | gum) | | | | | | | | | |
| 20 | API + | 50 | Ethanol | 3 | 1.0 ± 0.11 | 2.2 ± 0.07 | 3.8 ± 0.04 | 7.6 ± 0.17 | | |
| | XANTU | | (200% | | | | | | | |
| | RAL® | | Proof) | | | | | | | |
| | 75 | | | | | | | | | |
| | (xanthan | | | | | | | | | |
| | gum) | | | | | | | | | |
| Extraction of API Tablets in Water (Formulations 27-29) | | | | | | | | | | |
| 27 | API + | 50 | Water at | 5 | 1.05 ± 0.14 | 2.48 ± 0.16 | 4.33 ± 0.07 | 8.78 ± 0.26 | | |
| | GELCARI | | RT | | | | | | | |
| | N® GP | | | | | | | | | |
| | 379 | | | | | | | | | |
| | (carrageen | | | | | | | | | |
| | an) | | | | | | | | | |
| 28 | API + | 50 | Water at | 5 | 11.3 ± 1.74 | 27.3 ± 2.29 | 41.9 ± 2.59 | 71.6 ± 3.22 | | |
| | KLUCEL | | RT | | | | | | | |
| | ™ JXF | | | | | | | | | |
| | Pharm | | | | | | | | | |
| | (HPC) | | | | | | | | | |
| 29 | API + | 50 | Water at | 5 | 12.4 ± 0.69 | 23.8 ± 0.78 | 32.0 ± 0.89 | 48.0 ± 1.16 | | |
| | KLUCEL | | RT | | | | | | | |
| | ™ MF | | | | | | | | | |
| | Pharm | | | | | | | | | |
| | (HPC) | | | | | | | | | |
| Extraction of API Tablets in Ethanol (Formulations 27-29) | | | | | | | | | | |
| 27 | API + | 50 | Ethanol | 3 | 8.30 ± 0.13 | 13.38 ± 1.34 | 24.88 ± 3.08 | 40.27 ± 3.78 | | |
| | GELCARI | | (40% | | | | | | | |
| | N® GP | | Proof) | | | | | | | |
| | 379 | | | | | | | | | |
| | (carrageen | | | | | | | | | |
| | an) | | | | | | | | | |
| 28 | API + | 50 | Ethanol | 3 | 2.10 ± 0.18 | 7.40 ± 0.78 | 14.3 ± 1.04 | 28.1± 3.44 | | |
| | KLUCEL | | (40% | | | | | | | |
| | ™ JXF | | Proof) | | | | | | | |
| | Pharm | | | | | | | | | |
| | (HPC) | | | | | | | | | |
| 29 | API + | 50 | Ethanol | 3 | 2.20 ± 0.15 | 4.80 ± 0.31 | 6.50 ± 0.40 | 12.1 ± 0.84 | | |
| | KLUCEL | | (40% | | | | | | | |
| | ™ MF | | Proof) | | | | | | | |
| | Pharm | | | | | | | | | |
| | (HPC) | | | | | | | | | |
| | | | | Media | | | | | | |
| | | API | Dissolution | Volume | Percent Dissolved (Average ± SD) | | | | | |
| # | Compact | (mg) | Medium | (mL) | 15 min | 60 min | 120 min | | | |
| Extraction of API Tablets in Water (Formulations 36-40) | | | | | | | | | | |
| 36 | API + Na | 50 | Water at | 5 | 0.43 ± 0.07 | 1.19 ± 0.14 | 2.61 ± 0.32 | | | |
| | CMC | | RT | | | | | | | |
| 37 | API + | 50 | Water at | 5 | 8.5 ± 0.46 | 15.3 ± 0.93 | 25.8 ± 2.02 | | | |
| | PEO | | RT | | | | | | | |
| | 900 K | | | | | | | | | |
| | PEO | | | | | | | | | |
| | 400 K | | | | | | | | | |
| 38 | API + | 50 | Water at | 5 | 5.8 ± 0.51 | 10.4 ± 0.44 | 16.5 ± 0.41 | | | |
| | PEO | | RT | | | | | | | |
| | 900 K | | | | | | | | | |
| | XANTU | | | | | | | | | |
| | RAL® | | | | | | | | | |
| | 180 | | | | | | | | | |
| 39 | API + | 50 | Water at | 5 | 3.6 ± 0.06 | 8.1 ± 0.27 | 13.2 ± 0.36 | | | |
| | PEO | | RT | | | | | | | |
| | 900 K + | | | | | | | | | |
| | PROTA | | | | | | | | | |
| | NAL® | | | | | | | | | |
| | PH 6160 | | | | | | | | | |
| 40 | API + | 50 | Water at | 5 | 49 ± 0.23 | 9.7 ± 0.25 | 16.5 ± 0.21 | | | |
| | PEO | | RT | | | | | | | |
| | 900 K + | | | | | | | | | |
| | GELCA | | | | | | | | | |
| | RIN® | | | | | | | | | |
| | GP 379 | | | | | | | | | |
| Extraction of API Tablets in Ethanol (Formulations 36-40) | | | | | | | | | | |
| 36 | API + Na | 50 | Ethanol | 5 | 7.62 ± 0.12 | 13.94 ± 1.40 | 23.68 ± 1.96 | | | |
| | CMC | | (40% | | | | | | | |
| | | | Proof) | | | | | | | |
| 37 | API + | 50 | Ethanol | 5 | 10.6 ± 1.46 | 24.3 ± 5.99 | 41.2 ± 8.76 | | | |
| | PEO | | (40% | | | | | | | |
| | 900 K + | | Proof) | | | | | | | |
| | PEO | | | | | | | | | |
| | 400 K | | | | | | | | | |
| 38 | API + | 50 | Ethanol | 5 | 10.4 ± 0.63 | 32.7 ± 1.97 | 61.0 ± 1.34 | | | |
| | PEO | | (40% | | | | | | | |
| | 900 K | | Proof) | | | | | | | |
| | XANTU | | | | | | | | | |
| | RAL® | | | | | | | | | |
| | 180 | | | | | | | | | |
| 39 | API + | 50 | Ethanol | 5 | 17.9 ± 1.75 | 35.3 ± 2.60 | 57.1 ± 2.14 | | | |
| | PEO | | (40% | | | | | | | |
| | 900 K | | Proof) | | | | | | | |
| | PROTA | | | | | | | | | |
| | NAL® | | | | | | | | | |
| | PH 6160 | | | | | | | | | |
| 40 | API + | 50 | Ethanol | 5 | 12.0 ± 0.54 | 25.9 ± 2.49 | 43.5 ± 3.13 | | | |
| | PEO | | (40% | | | | | | | |
| | 900 K | | Proof) | | | | | | | |
| | GELCA | | | | | | | | | |
| | RIN® | | | | | | | | | |
| | GP 379 | | | | | | | | | |
| | | | | Media | | | | | | |
| | | API | Dissolution | Volume | Percent Dissolved (Average ± SD) | | | | | |
| # | Compact | (mg) | Medium | (mL) | 15 min | 60 min | 120 min | 240 min | | |
| Extraction of API Tablets in Ethanol (Formulations 14, 16-19, 27, 29 and 36) | | | | | | | | | | |
| 14 | API + | 50 | Ethanol | 3 | 4.64 ± 0.75 | 11.60 ± 0.79 | 17.97 ± 1.41 | 30.43 ± 3.58 | | |
| | PEO | | (40% | | | | | | | |
| | (900 K) | | Proof) | | | | | | | |
| 16 | API + | 50 | Ethanol | 3 | 5.6 ± 0.52 | 12.5 ± 0.14 | 18.9 ± 0.52 | \26.4 ± 1.25 | | |
| | PEO (8 | | (40% | | | | | | | |
| | million) | | Proof) | | | | | | | |
| 17 | API + | 50 | Ethanol | 3 | 6.1 ± 0.26 | 14.2 ± 0.37 | 24.0 ± 0.49 | 36.1 ± 1.10 | | |
| | HPMC | | (40% | | | | | | | |
| | Kl00 M | | Proof) | | | | | | | |
| 18 | API + | 50 | Ethanol | 3 | 23.5 ± 1.20 | 50.0 ± 3.61 | 71.8 ± 6.73 | 81.5 ± 6.33 | | |
| | PROTAN | | (40% | | | | | | | |
| | AL® PH | | Proof) | | | | | | | |
| | 6160 | | | | | | | | | |
| | (Sodium | | | | | | | | | |
| | Alginate) | | | | | | | | | |
| 19 | API + | 50 | Ethanol | 3 | 2.3 ± 0.43 | 4.5 ± 0.79 | 6.8 ± 0.96 | 10.6 ± 1.66 | | |
| | XANTUR | | (40% | | | | | | | |
| | AL® 180 | | Proof) | | | | | | | |
| | (xanthan | | | | | | | | | |
| | gum) | | | | | | | | | |
| 27 | API + | 50 | Ethanol | 3 | 3.9 ± 0.62 | 8.7 ± 0.20 | 13.9 ± 2.92 | 23.5 ± 1.05 | | |
| | GELCARI | | (40% | | | | | | | |
| | N® GP | | Proof) | | | | | | | |
| | 379 | | | | | | | | | |
| | (carrageen | | | | | | | | | |
| | an) | | | | | | | | | |
| 29 | API + | 50 | Ethanol | 3 | 10.8 ± 1.05 | 22.2 ± 0.77 | 29.7 ± 0.96 | 38.6 ± 2.22 | | |
| | KLUCEL | | (40% | | | | | | | |
| | ™ MF | | Proof) | | | | | | | |
| | (HPC) | | | | | | | | | |
| 36 | API + Na | 50 | Ethanol | 3 | 1.5 ± 0.24 | 2.3 ± 0.23 | 4.0 ± 0.57 | 6.2 ± 0.53 | | |
| | CMC | | (40% | | | | | | | |
| | | | Proof) | | | | | | | |
| | | | | Media | | | | | | |
| | | API | Dissolution | Volume | Percent Dissolved (Average ± SD) | | | | | |
| # | Compact | (mg) | Medium | (mL) | 15 min | 30 min | 45 min | 60 min | 90 min | 120 min |

Extraction of API From Non-High Viscosity Formulation (REF, See Example 6) Tablets in Water and Ethanol

| | | | | | | | | | | |
|---|---|---|---|---|---|---|---|---|---|---|
| REF | API | 200 | Water at | 5 | 6.71 ± 5.92 | 12.19 ± 2.06 | 20.43 ± 6.12 | 29.2 ± 3.48 | 39.20 ± 3.60 | 57.21 ± 15.63 |
| | Tablets, | | RT | | | | | | | |
| | 200 mg | | | | | | | | | |
| REF | API | 200 | Ethanol | 5 | 0.7 ± 0.02 | 6.6 ± 0.51 | 15.2 ± 6.64 | 21.3 ± 3.85 | 27.2 ± 1.19 | 29.0 ± 0.78 |
| | Tablets, | | (40% | | | | | | | |
| | 200 mg | | Proof) | | | | | | | |

Extraction of API Tablets in Water (Formulations 43-46)

| | | | | | | | | | | |
|---|---|---|---|---|---|---|---|---|---|---|
| 43 | API + (Trial | 50 | Water at | 3 | 12.07 ± 0.76 | 21.47 ± 1.32 | 26.98 ± 1.29 | 36.50 ± 1.88 | 45.42 ± 2.02 | 52.04 ± 2.15 |
| | D Powder) | | RT | | | | | | | |
| | + PEO | | | | | | | | | |
| | 900 K | | | | | | | | | |
| 44 | API + (Trial | 50 | Water at | 3 | 5.6 ± 0.47 | 8.9 ± 1.48 | 12.5 ± 0.43 | 15.3 ± 0.27 | 20.9 ± 0.64 | 24.8 ± 0.32 |
| | D Powder) | | RT | | | | | | | |
| | + Na | | | | | | | | | |
| | CMC | | | | | | | | | |
| 45 | API + (Trial | 50 | Water at | 3 | 7.1 ± 1.34 | 10.5 ± 0.57 | 13.8 ± 1.07 | 17.2 ± 1.14 | 22.8 ± 1.68 | 26.5 ± 1.16 |
| | D Powder) | | RT | | | | | | | |
| | + | | | | | | | | | |
| | XANTUR | | | | | | | | | |
| | AL® 180 | | | | | | | | | |
| | (xanthan | | | | | | | | | |
| | gum) | | | | | | | | | |
| 46 | API + (Trial | 50 | Water at | 3 | 6.9 ± 1.62 | 11.6 ± 1.61 | 14.7 ± 0.51 | 18.9 ± 1.81 | 24.4 ± 1.30 | 28.5 ± 1.92 |
| | D Powder) | | RT | | | | | | | |
| | + | | | | | | | | | |
| | GELCARI | | | | | | | | | |
| | N® GP | | | | | | | | | |
| | 379 | | | | | | | | | |
| | (carrageen | | | | | | | | | |
| | an) | | | | | | | | | |

Extraction of API Tablets in Ethanol (Formulations 43-46)

| | | | | | | | | | | |
|---|---|---|---|---|---|---|---|---|---|---|
| 43 | API + (Trial | 50 | Ethanol | | | | | | | |
| | D Powder) | | (40% | 3 | 5.56 ± 0.36 | 11.10 ± 0.74 | 15.04 ± 0.19 | 19.70 ± 0.63 | 25.76 ± 0.63 | 30.37 ± 0.60 |
| | +PEO | | Proof) | | | | | | | |
| | 900 K | | | | | | | | | |
| 44 | API + (Trial | 50 | Ethanol | 3 | 1.6 ± 0.55 | 3.0 ± 0.95 | 6.5 ± 2.34 | 10.3 ± 1.84 | 16.0 ± 3.37 | 18.0 ± 3.03 |
| | D Powder) | | (40% | | | | | | | |
| | + Na | | Proof) | | | | | | | |
| | CMC | | | | | | | | | |
| 45 | API + (Trial | 50 | Ethanol | 3 | 4.0 ± 0.33 | 5.2 ± 0.24 | 6.3 ± 0.61 | 8.6 ± 0.18 | 11.5 ± 1.05 | 14.2 ± 0.74 |
| | D Powder) | | (40% | | | | | | | |
| | + | | Proof) | | | | | | | |
| | XANTUR | | | | | | | | | |
| | AL® 180 | | | | | | | | | |
| | (xanthan | | | | | | | | | |
| | gum) | | | | | | | | | |
| 46 | API + (Trial | 50 | Ethanol | 3 | 0.9 ± 0.07 | 2.1 ± 0.18 | 3.1 ± 0.09 | 4.7 ± 0.15 | 7.1 ± 0.25 | 9.4 ± 0.29 |
| | D Powder) | | (40% | | | | | | | |
| | + | | Proof) | | | | | | | |
| | GELCARI | | | | | | | | | |
| | N® GP | | | | | | | | | |
| | 379 | | | | | | | | | |
| | (carrageen | | | | | | | | | |
| | an) | | | | | | | | | |

Extraction of API Powder Blend in Water (Formulations 43-46)

| | | | | | | | | | | |
|---|---|---|---|---|---|---|---|---|---|---|
| 43 | API (Trial | 50 | Water at | 3 | 43.61 ± 4.27 | 53.65 ± 5.15 | 58.47 ± 3.19 | 64.73 ± 4.14 | 70.83 ± 5.44 | 75.02 ± 3.62 |
| | D Powder) | | RT | | | | | | | |
| | + PEO | | | | | | | | | |
| | 900 K | | | | | | | | | |
| 44 | API + (Trial | 50 | Water at | 3 | 35.6 ± 9.53 | 40.7 ± 9.16 | 33.1 ± 4.45 | 33.6 ± 2.70 | 40.7 ± 2.95 | 40.9 ± 4.79 |
| | D Powder) | | RT | | | | | | | |
| | + Na | | | | | | | | | |
| | CMC | | | | | | | | | |
| 45 | API + (Trial | 50 | Water at | 3 | 14.7 ± 2.28 | 24.1 ± 3.90 | 24.5 ± 1.48 | 27.7 ± 2.70 | 34.9 ± 5.94 | 38.3 ± 5.49 |
| | D Powder) | | RT | | | | | | | |
| | + | | | | | | | | | |
| | XANTUR | | | | | | | | | |
| | AL® 180 | | | | | | | | | |
| | (xanthan | | | | | | | | | |
| | gum) | | | | | | | | | |
| 46 | API + (Trial | 50 | Water at | 3 | 39.1 ± 7.30 | 48.7 ± 12.66 | 45.6 ± 7.19 | 52.8 ± 7.98 | 55.3 ± 7.66 | 56.2 ± 10.75 |
| | D Powder) | | RT | | | | | | | |
| | + | | | | | | | | | |
| | GELCARI | | | | | | | | | |
| | N® GP | | | | | | | | | |
| | 379 | | | | | | | | | |
| | (carrageen | | | | | | | | | |
| | an) | | | | | | | | | |

Extraction of API Powder Blend in Ethanol (Formulations 43-46)

| | | | | | | | | | | |
|---|---|---|---|---|---|---|---|---|---|---|
| 43 | API (Trial | 50 | Ethanol | 3 | 33.23 ± 6.87 | 36.47 ± 6.00 | 40.31 ± 5.96 | 42.47 ± 6.89 | 46.90 ± 7.40 | 49.71 ± 7.10 |
| | D Powder) | | (40% | | | | | | | |
| | + PEO | | Proof) | | | | | | | |
| | 900 K | | | | | | | | | |
| 44 | API + (Trial | 50 | Ethanol | 3 | 15.0 ± 10.86 | 13.6 ± 8.05 | 14.3 ± 6.35 | 15.2 ± 7.31 | 19.0 ± 9.19 | 20.1 ± 8.99 |
| | D Powder) | | (40% | | | | | | | |
| | + Na | | Proof) | | | | | | | |
| | CMC | | | | | | | | | |
| 45 | API + (Trial | 50 | Ethanol | 3 | 14.2 ± 1.67 | 21.2 ± 7.67 | 21.2 ± 2.68 | 25.7 ± 9.61 | 29.7 ± 7.03 | 26.5 ± 7.53 |
| | D Powder) | | (40% | | | | | | | |
| | + | | Proof) | | | | | | | |
| | XANTUR | | | | | | | | | |
| | AL® 180 | | | | | | | | | |
| | (xanthan | | | | | | | | | |
| | gum) | | | | | | | | | |
| 46 | API + (Trial | 50 | Ethanol | 3 | 30.1 ± 14.71 | 33.6 ± 14.57 | 40.6 ± 16.63 | 37.6 ± 15.42 | 37.3 ± 13.94 | 40.0 ± 14.78 |
| | D Powder) | | (40% | | | | | | | |
| | + | | Proof) | | | | | | | |
| | GELCARI | | | | | | | | | |
| | N® GP | | | | | | | | | |
| | 379 | | | | | | | | | |
| | (carrageen | | | | | | | | | |
| | an) | | | | | | | | | |

### Example 3

### Further Oral Tablet Formulations

Oral tablet formulations were prepared according to the following manufacturing methodology.

Dispensing—materials were weighed and dispensed in separate poly bags.

Pre blending and screening—the active ingredient and all the other intra granular excipients were charged in a diffusive blender and the contents blended to form a pre blend. The pre blend was screened through a screen mill. The screened materials were collected in separate poly bags.

Preparation of povidone granulation solution—purified water was charged to a suitable vessel. Povidone was added and mixed until dissolved.

Granulation & Drying—the screened pre blend was charged into a fluid bed granulator. The contents were granulated using the povidone solution. The granules were dried in a fluid bed dryer, until a moisture content value of less than 2.5% was achieved using a moisture balance set at 105° C.

Milling—the dried granules were milled through a screen using a screen mill.

Blending—the weights of the extra-granular ingredients were adjusted based on the yield of granules to achieve the target tablet formula. The extra-granular ingredients were screened through a screen. The milled granules and extra-granular ingredients were charged to a diffusion blender and the contents blended. Magnesium stearate was added to the diffusion blender and the contents mixed. The blend was discharged and reconciled.

Compression—the blend was compressed on a rotary tablet press using appropriate tooling (punches and die), to target weight. Friability was checked at the beginning of the compression run, and periodically thereafter, for individual tablet weights, thickness and hardness.

Coating—purified water was charged. The coating material was added and mixed until uniformly dispersed in purified water. The tablets were spray-coated in a pan coater to a weight gain of not less than 4% w/w. The tablets were cooled to room temperature and discharged from the coating pan.

Formulations 1-4 were prepared according to the manufacturing methodology described in this Example.

| | | | | |
|---|---|---|---|---|
| | #1 | #2 | #3 | #4 |
| Ingredient | (mg/tablet) | (mg/tablet) | (mg/tablet) | (mg/tablet) |

Intra-granular

| | | | | |
|---|---|---|---|---|
| α-6-mPEG₆-O-oxycodol | 232.48¹ | 232.48¹ | 232.48¹ | 232.48¹ |
| phosphate | | | | |
| Xanthan Gum | N/A | 30 | 30 | 45 |

Extra-Granular

| | | | | |
|---|---|---|---|---|
| NaCMC | 80.00 | 60.00 | N/A | N/A |
| Xanthan Gum | N/A | N/A | 60.00 | 40.00 |
| Core Tablet Weight | 800.00 | 800.00 | 800.00 | 800.00 |

Film Coating

| | | | | |
|---|---|---|---|---|
| Opadry II 85F18520 | 32.00 | 32.00 | 32.00 | 32.00 |
| White | | | | |
| Purified Water² | q.s. | q.s. | q.s. | q.s. |
| Film Coated Tablet | 832.00 | 832.00 | 832.00 | 832.00 |
| Weight | | | | |

¹Equivalent to 200 mg of α-6-mPEG₆-O-oxycodol
²Removed during processing

Formulations 5-9 were prepared according to the manufacturing methodology described in this Example.

| | | | | | |
|---|---|---|---|---|---|
| | #5 | #6 | #7 | #8 | #9 |
| Ingredient | (mg/tablet) | (mg/tablet) | (mg/tablet) | (mg/tablet) | (mg/tablet) |

Intra-granular

| | | | | | |
|---|---|---|---|---|---|
| α-6-mPEG₆-O-oxycodol phosphate | 232.48¹ | 232.48¹ | 232.48¹ | 232.48¹ | 232.48¹ |

Extra-Granular

| | | | | | |
|---|---|---|---|---|---|
| Xanthan Gum | 40.00-160.00 | N/A | 40.00-80.00 | 40.00-80.00 | N/A |
| NaCMC | N/A | N/A | 40.00-80.00 | N/A | 40.00-80.00 |
| Polyethylene Oxide | N/A | 40.00-160.00 | N/A | 40.00-80.00 | 40.00-80.00 |
| Core Tablet Weight | 800.00 | 800.00 | 800.00 | 800.00 | 800.00 |

Film Coating

| | | | | | |
|---|---|---|---|---|---|
| Opadry II 85F18520 White | 32.00 | 32.00 | 32.00 | 32.00 | 32.00 |
| Purified Water² | q.s. | q.s. | q.s. | q.s. | q.s. |
| Film Coated Tablet Weight | 832.00 | 832.00 | 832.00 | 832.00 | 832.00 |

¹Equivalent to 200 mg of α-6-mPEG₆-O-oxycodol
²Removed during processing

Formulations A1-A3 were prepared according to the manufacturing methodology described in this Example.

| | | | |
|---|---|---|---|
| | Formulation | Formulation | Formulation |
| Ingredient | A1 | A2 | A3 |

Intra-granular

| | | | |
|---|---|---|---|
| α-6-mPEG₆-O-oxycodol | 232.48 | 232.48 | 232.48 |
| phosphate | | | |
| Xanthan gum | N/A | 45 | 30 |
| (XANTURAL ® 180) | | | |

Extra-Granular

| | | | |
|---|---|---|---|
| Sodium carboxymethyl | 80.00 | N/A | 60.00 |
| cellulose (7HXF) | | | |
| Xanthan gum | N/A | 40.00 | N/A |
| (XANTURAL ® 180) | | | |
| Core Tablet Weight | 800.00 | 800.00 | 800.00 |

Formulation A1 Contains 10% sodium carboxymethylcelluose (NaCMC)
Formulation A2 Contains 10.6% xanthan gum (XG)
Formulation A3 Contains 3.75% XG +7 .5% NaCMC

Formulation A4 was prepared according to the manufacturing methodology described in this Example.

Formulation A4

| | |
|---|---|
| Ingredient | Mg/tablet |

Intra-granular

| | |
|---|---|
| α-6-mPEG₆-O-oxycodol phosphate | 232.48 |

Extra-Granular

| | |
|---|---|
| Sodium carboxymethyl cellulose (7HXF) | 60.00 |
| Core Tablet Weight | 800.00 |

Formulation A4 Contains 7.5% Sodium Carboxy Methyl Cellulose (NaCMC)

Formulations including an antioxidant were prepared according to the manufacturing methodology described in this Example.

10% NaCMC formulation with anti-oxidant (Propyl gallate)

| | |
|---|---|
| Ingredients | Mg/tablet |

Intra granular Part

| | |
|---|---|
| α-6-mPEG₆-O-oxycodol phosphate | 232.480 |

Binder Solution

| | |
|---|---|
| Propyl gallate (Minakem) | 0.500 |
| Purified water | q.s. |
| Granule Total | 600.000 |

Extra granular Part

| | |
|---|---|
| Sodium CMC (AQUALON ® CMC 7HXF) | 80.00 |
| Core tablet weight | 800.00 |

### Example 4

### Extraction Assay

An extraction assay was conducted to determine the amount of active agent extractable from the formulations. Briefly, the formulations were cut into four pieces, placed into 10 mL of water, and agitated for 30 minutes at room temperature or elevated temperature. The amount of active agent extracted was measured by HPLC with UV detection.

The amount of active agent extracted is provided in the following table.

Amount of Active Agent Extracted

| | Amount of α-6-mPEG₆-O-oxycodol | |
|---|---|---|
| | phosphate extracted (mg) | |
| | | at Elevated |
| | at Room | Temperature |
| Formulations | Temperature | 95° C. |
| Formulation # 1 | 12.78 | 40.38 |
| Formulation # 2 | 14.50 | 33.68 |
| Formulation # 3 | 12.96 | 43.04 |
| Formulation # 4 | 12.46 | 73.64 |

Results from Formulation A4 (7.5% NaCMC) extraction at room temperature assay are provided in the following table.

| Amount Extracted | Standard | Percent amount extracted |
|---|---|---|
| (mg/ml) | Deviation | of dose strength |
| Average of n = 3 | (SD) | (200 mg) |
| 10.06 | 1.44 | 5% |

As can be seen from these, and other, results, in neutral medium (e.g., pH 7) and in water, NaCMC is believed to be unionized and the mechanism of active agent release from polymer matrix is believed to be different compared to the polymer in an acidic medium. At neutral pH and in water, the rate of active agent release is slower. This property is helpful in retarding extraction in small volumes of water over a shorter time period and therefore is good for abuse deterrence.

### Example 5

### Dissolution Assay

A dissolution assay was conducted to determine the amount of active agent released from the formulations. Briefly, the formulations were dissolved in a dissolution media (0.1N HCl, pH 1.2, 900 mL volume) and placed into a USP Dissolution Apparatus 2 at 75 rpm. The amount of active agent released was measured by HPLC using the following parameters.

Dissolution Testing HPLC Parameters

| | |
|---|---|
| Instrument | Waters 2695 System |
| Column | Agilent ZORBAX ® 300 SB-C₁₈, 50 × 4.6 mm, 3.5 μm |
| Mobile | Isocratic-(20:80) Acetonitrile (can): H₂O Plus 0.1% (v/v) |
| phase | trifluoroacetic acid (TFA) |
| Diluent | Stock standard solution: (20:80) ACN: H₂O Plus 0.1% (v/v) |
| | TFA Working standard solution: Dissolution media |

The percentage of active agent released is provided in the following tables.

Percentage of Active Agent Released for Formulation #1

| | Percentage of active agent released | | | | | | |
|---|---|---|---|---|---|---|---|
| | 5 min | 10 min | 15 min | 30 min | 45 min | 60 min | Inf* |
| Average | 62.6 | 102.1 | 102.4 | 102.4 | 102.8 | 102.9 | 103.2 |
| Standard Deviation | 2.41 | 1.48 | 1.71 | 1.42 | 1.70 | 1.54 | 1.55 |
| Relative Standard Deviation | 3.85 | 1.45 | 1.67 | 1.39 | 1.65 | 1.50 | 1.50 |

Percentage of Active Agent Released for Formulation #2

| | Percentage of active agent released | | | | | | |
|---|---|---|---|---|---|---|---|
| | 5 min | 10 min | 15 min | 30 min | 45 min | 60 min | Inf* |
| Average | 44.2 | 91.5 | 102.9 | 103.0 | 103.4 | 103.5 | 103.7 |
| Standard Deviation | 5.39 | 3.15 | 1.10 | 1.11 | 1.27 | 1.21 | 1.32 |
| Relative Standard Deviation | 12.21 | 3.45 | 1.07 | 1.08 | 1.23 | 1.17 | 1.27 |

Percentage of Active Agent Released for Formulation #3

| | Percentage of active agent released | | | | | | |
|---|---|---|---|---|---|---|---|
| | 5 min | 10 min | 15 min | 30 min | 45 min | 60 min | Inf* |
| Average | 14.8 | 27.8 | 35.2 | 50.4 | 72.3 | 97.0 | 105.0 |
| Standard Deviation | 1.47 | 2.42 | 3.05 | 5.73 | 16.67 | 6.43 | 1.84 |
| Relative Standard Deviation | 9.96 | 8.71 | 8.67 | 11.37 | 23.07 | 6.62 | 1.75 |

Percentage of Active Agent Released for Formulation #6

| | Percentage of active agent released | | | | | | |
|---|---|---|---|---|---|---|---|
| | 5 min | 10 min | 15 min | 30 min | 45 min | 60 min | Inf* |
| Average | 11.1 | 26.3 | 37.1 | 57.3 | 80.2 | 99.2 | 104.1 |
| Standard Deviation | 0.50 | 1.30 | 1.93 | 3.87 | 9.43 | 2.62 | 1.13 |
| Relative Standard Deviation | 4.53 | 4.95 | 5.21 | 6.75 | 11.76 | 2.65 | 1.09 |

The active agent in the formulations were rapidly and effectively released in a dissolution medium having low pH (e.g., pH of 1.2). As the pH of gastric acid is generally about 1.5 to 3.5, these formulations should rapidly and effectively release the active agent upon oral administration.

As can be seen from these results and other results, the active agent is readily released for certain formulations comprising NaCMC, in acidic medium (at pH 1-2, similar to that of the stomach). Without being limited as to theory, NaCMC is believed to be ionized and hence drug release from the formulations is rapid in acidic medium, with more than 85% of the active agent released in 15 minutes. Thus, these formulations have release kinetics similar to immediate release formulations.

### Example 6

### Pharmacokinetics Study

The pharmacokinetic profiles of high viscosity formulations with varying NaCMC content (BE1 and BE2) were compared to a reference non-high viscosity formulation (REF). The REF formulation was a 200 mg tablet comprising 232.40 mg α-6-mPEG₆-O-oxycodol phosphate (equivalent to 200 mg) in an intra-granular portion of the tablet. The tablet core was 800 mg and included a film coating comprised of Opadry II 85F18520 White. The BE1 and BE2 formulations are provided below.

### Higher and Lower NaCMC Tablet Formulations

| | | | |
|---|---|---|---|
| | | Formulation | Formulation |
| | | BE1 | BE2 |
| | Compendial | 10% NaCMC | 7.5% NaCMC |
| Ingredient | Grade | (mg/tablet) | (mg/tablet) |

Intra-granular

| | | | |
|---|---|---|---|
| α-6-mPEG₆-O-oxycodol phosphate | Not Applicable | 232.48¹ | 232.48¹ |
| Microcrystalline cellulose | NF, Ph. Eur. | 193.87 | 193.87 |
| (Avicel PH 101) | | | |
| Dibasic calcium phosphate anhydrous | USP, Ph. Eur. | 128.75 | 128.75 |
| (Fujicalin) | | | |
| Croscarmellose sodium (AC-DI-SOL ®) | NF, Ph. Eur. | 13.20 | 13.20 |
| Colloidal silicon dioxide | NF, Ph. Eur. | 21.00 | 21.00 |
| (CAB-O-SIL ® M5P) | | | |
| Povidone (PLASDONE ™ K-29/32) | USP, Ph. Eur. | 10.20 | 10.20 |
| Propyl gallate | USP/NF | 0.50 | 0.50 |
| Purified Water^{b} | USP | q.s. | q.s. |

Extra-Granular

| | | | |
|---|---|---|---|
| Sodium carboxymethyl cellulose | USP/NF | 80.00 | 60.00 |
| (AQUALON ® 7HXF) | | | |
| Microcrystalline cellulose | NF, Ph. Eur. | 104.00 | 124.00 |
| (Avicel PH 102) | | | |
| Colloidal silicon dioxide | NF, Ph. Eur. | 8.00 | 8.00 |
| (CAB-O-SIL ® MSP) | | | |
| Magnesium stearate | NF, Ph. Eur. | 8.00 | 8.00 |
| Core Tablet Weight (mg) | | 800.00 | 800.00 |

Film Coating

| | | | |
|---|---|---|---|
| Opadry II 85F18520 White | Proprietary | 32.00 | 32.00 |
| Purified Water² | USP | q.s. | q.s. |
| Film Coated Tablet Weight (mg) | | 832.00 | 832.00 |

¹232.48 mg is equivalent to 200 mg of α-6-mPEG₆-O-oxycodol.
²Removed during processing.

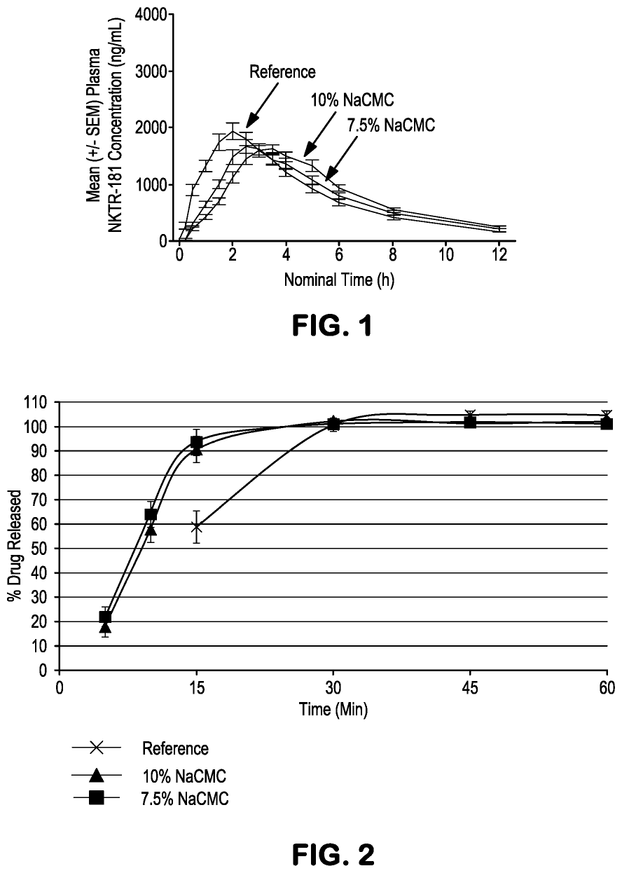

A conventional randomized and crossover study in healthy human male and female subjects was conducted to evaluate the pharmacokinetic profiles of a single 232.48 mg dose of α-6-mPEG₆-O-oxycodol phosphate (equivalent to 200 mg dose of α-6-mPEG₆-O-oxycodol) administered orally under fasted conditions (overnight fast of minimum 10 hours and continuing fast for 4 hours post-dosing) as Formulations BE1, BE2, and REF. Subjects were randomized to one of six different treatment sequences of 200 mg of BE1, BE2, and REF based on a William's design consisting of two Latin squares. There were seven days between dose administrations. Blood samples were obtained pre-dose and at multiple time points over 72-hours post-dose. The blood samples were assayed for the concentration of α-6-mPEG₆-O-oxycodol. The rate (peak concentration, Cₘₐₓ) and extent of absorption (area under the concentration-time curve, AUCₗₐₛₜ and AUC_{inf}) were calculated for BE1, BE2, and REF. FIG. 1 shows the mean plasma concentration for the active agent over time. The ratios of the geometric least-square (LS) means of BE1 and BE2 with respect to REF and associated two-sided 90% confidence interval (CI) for Cₘₐₓ and AUC were calculated.

To compare the rate and extent of α-6-mPEG₆-O-oxycodol phosphate absorption between treatments, a mixed-effect model with log transformed PK parameters was used to estimate the ratios of geometric least-squares (LS) means between BE1, BE2, and REF and associated two sided 90% confidence intervals (CIs) for Cₘₐₓ and AUC. The model had treatment, period, and sequence as fixed effects, and subject nested in the sequence as a random effect. The two-sided 90% confidence intervals for the ratio of geometric Least Square Means for BE1, BE2, and REF are presented in the table below.

### Pharmacokinetic Parameters in Human Subjects

Percent of REF at 90% CI (Low-High)

| | Formulation | Formulation |
|---|---|---|
| Parameter | BE1 | BE2 |
| Cₘₐₓ | 81-99 | 82-100 |
| AUCₗₐₛₜ | 101-110 | 97-106 |
| AUC_{inf} | 101-110 | 97-106 |

Cₘₐₓ: Maximum observed plasma concentration.
AUCₗₐₛₜ: Area under the plasma concentration-time curve from time = 0 to the time of the last measureable concentration.
AUC_{inf}: Area under the plasma concentration-time curve from time = 0 to infinity.

The mean Cₘₐₓ values of the 7.5% and the 10% NaCMC formulations were slightly lower than those of the REF formulation. The mean AUC₀₋ₗₐₛₜ and AUC_{0-inf} values for the 7.5% and 10% NaCMC formulations were comparable to those of the REF formulation. The mean Tₘₐₓ of the 10% NaCMC formulation was approximately one hour greater than the REF formulation. The mean Tₘₐₓ value of the 7.5% NaCMC formulation was 0.6 hours greater than the mean Tₘₐₓ of the REF formulation. The mean terminal half-life values for the 10% and 7.5% NaCMC formulations (9.6 and 11.33 hours, respectively) were similar to the REF formulation (10.38 hours).

To assess bioequivalence, Cₘₐₓ, AUC₀₋ₗₐₛₜ, and AUC_{0-inf} values were analyzed by linear mixed effect models. As seen in the table above, the 90% confidence interval (CI) for the geometric mean test/reference formulation ratios for Cₘₐₓ, AUC₀₋ₗₐₛₜ, and AUC_{0-inf} were all contained within bioequivalence limits of 80% to 125% indicating the 7.5% and 10% NaCMC formulations were both bioequivalent to the REF formulation.

### Example 7

### Syringe Extraction Assay

The syringe-ability of extracts of intact and ground reference formulation and three high viscosity formulations were tested.

Methods

A low viscosity formulation as well as high viscosity formulations P1, P2, and P2 were prepared, either intact or ground by coffee grinder for 60 seconds.

Low Viscosity Formulation:

| | | |
|---|---|---|
| | Compendial | Amount |
| Ingredient | Grade | (mg/tablet) |

Intra-granular

| | | |
|---|---|---|
| α-6-mPEG₆-O-oxycodol phosphate | Not Applicable | 232.48¹ |
| Core Tablet Weight | | 800.00 |

Film Coating

| | | |
|---|---|---|
| Opadry II 85F18520 White | Proprietary | 32.00 |
| Purified Water² | USP | q.s. |
| Film Coated Tablet Weight | | 832.00 |

¹232.48 mg of α-6-mPEG₆-O-oxycodol phosphate is equivalent to 200 mg of α-6-mPEG₆-O-oxycodol.
²Removed during processing.

Formulation P1:

| | | |
|---|---|---|
| | | Quantity |
| Material | Brand Name/Supplier | (mg per tablet) |

Intragranular:

| | |
|---|---|
| α-6-mPEG₆-O-oxycodol | 232.480¹ |
| Phosphate | |
| Granules Total | 600.000 |

Extragranular:

| | | |
|---|---|---|
| Sodium Carboxy Methyl | AQUALON ®/Ashland | 80.000 |
| Cellulose, USP | | |
| Total | | 800.000 |

Coating:

| | | |
|---|---|---|
| Opadry II White 85F18520 | OPADRY ®/Colorcon | 32.000 |
| (12% Titanium Dioxide) | | |
| Purified water USP² | Ricca Chemicals/ | Q.S. |

| | |
|---|---|
| Coated Tablet Weight (mg) | 832.000 |

¹232.48 mg of α-6-mPEG₆-O-oxycodol phosphate is equivalent to 200 mg of α-6-mPEG₆-O-oxycodol.
²Removed during processing.

Formulation P2:

| | | |
|---|---|---|
| | | Quantity |
| Material | Brand Name/Supplier | (mg per tablet) |

Intragranular:

| | |
|---|---|
| α-6-mPEG₆-O-oxycodol | 232.480¹ |
| Phosphate | |

| | |
|---|---|
| Granules Total | 600.000 |

Extragranular:

| | | |
|---|---|---|
| Sodium Carboxy Methyl | AQUALON ®/Ashland | 60.000 |
| Cellulose, USP | | |

| | |
|---|---|
| Total | 800.000 |

Coating:

| | | |
|---|---|---|
| OPADRY ® II White | OPADRY ®/Colorcon | |
| 85F18520 | | 32.000 |
| (12% Titanium Dioxide) | | |
| Purified water USP² | Ricca Chemicals/ | Q.S. |

| | |
|---|---|
| Coated Tablet Weight (mg) | 832.000 |

¹232.48 mg of α-6-mPEG₆-O-oxycodol phosphate is equivalent to 200 mg of α-6-mPEG₆-O-oxycodol.
²Removed during processing.

Formulation P3:

| | | |
|---|---|---|
| | | Quantity |
| Material | Brand Name/Supplier | (mg per tablet) |

Intragranular:

| | | |
|---|---|---|
| α-6-mPEG₆-O-oxycodol | | 232.480¹ |
| Phosphate | | |
| Xanthan Gum, USP | XANTURAL ® 180/ | 8.000 |
| | CP Kelco | |

| | |
|---|---|
| Granules Total | 600.000 |

Extragranular:

| Sodium Carboxy Methyl | Ashland/ | 60.000 |
|---|---|---|
| Cellulose, USP | | |
| Total | | 800.000 |
| Coating: | | |
| OPADRY ® II White | OPADRY ®/Colorcon | |
| 85F18520 | | 32.000 |
| (12% Titanium Dioxide) | | |
| Purified water USP² | Ricca Chemicals/ | Q.S. |

| | |
|---|---|
| Coated Tablet Weight (mg) | 832.000 |

¹232.48 mg of α-6-mPEG6ₙ-O-oxycodol phosphate is equivalent to 200 mg of α-6-mPEG₆-O-oxycodol.
²Removed during processing.

For experiments with intact tablets, the average mass of an intact tablet was recorded by weighing 18 tablets from each formulation; for experiments with ground tablets, the recovered weight of each ground tablet was recorded. 10 mL of tap water was added to each sample in a 20 mL glass vial. For ground extractions, once the solvent was added, the extract was vortexed for 10 seconds to ensure all ground material was wetted—the vortexing was repeated up to three times total. Tablets were extracted over the specified time (30 minutes), without agitation or with agitation at 200 rpm in a laboratory orbital shaker. Syringe barrels (10 mL) were fitted with either 18 or 22 gauge 1½ inch needles, and the empty syringe weight recorded. To avoid clogging of the needle, liquid was drawn through a cigarette filter after the needle was fitted with a modified needle protector to act as a guide preventing the needle from protruding through the filter. The needle, with the filter attached, was allowed to soak in the extract for ten seconds, and an attempt was made to draw up extract from the extraction vial for a maximum time of three minutes.

The volume of extract drawn into the syringe was recorded (using syringe graduations). The cigarette filter was discarded immediately following the draw attempt made at room temperature, or after the syringe with heated content was allowed to cool down to approximate body temperature as judged by touch. The weight of loaded syringe was recorded.

Material was attempted to be expelled from the needle into a pre-weighed 20 mL glass vial, for a maximum of 3 minute or until resistance caused the hand to shake. The gross weight of the 20 mL glass vial with the expelled material was recorded and the weight of expelled material was calculated.

All sample weights, volume and weight of expelled liquid, as well as observations, were recorded.

If the ejected weight was ≥1.0 gram (≥10% of the water volume used considering 1 mL is equivalent to 1 gram) which is the minimal volume needed for the viscometer, a viscosity measurement was performed on the expelled extract. The viscosity analysis was performed using a water-jacketed measurement cell, designed to maintain constant temperature during the analysis. Viscosity was measured at the lab ambient temperature, which was maintained in the range of 66 to 77° F. (19 to 25° C.). When the ≥10% criterion was achieved, α-6-mPEG₆-O-oxycodol content was measured by LC-MS/MS.

The following observations were recorded: Volume and weight drawn into the syringe using 18G and 22G needles; Visible appearance of extract; Weight of any extract expelled from the syringe; Viscosity of any extract expelled from the syringe if weight expelled was greater than 10% of extract weight; Percent α-6-mPEG₆-O-oxycodol recovered from each expelled fraction if weight expelled was greater than 10% of extract weight.

From these observations, the average weight drawn, average weight expelled, and average percent α-6-mPEG₆-O-oxycodol recovered were calculated.

Results:

Syringe-able Extract Weight: For all conditions tested on the Low Viscosity formulation, about 66% to 79% of the extract was syringe-able (drawn into syringe). There was no significant impact of any of the test conditions on syringe-ability (tablet—intact or ground, agitation—shaking or no-shaking, temperature—room temperature (RT) or 90° C., or syringe needle sizes—18G or 22G).

For high viscosity formulations P1, P2, and P3, at room temperature, the percent syringe-able extract weights for formulations P1, P2, and P3 (shaking or no-shaking, and 18G or 22 G syringe needles) ranged from about 49% to 79%. At elevated temperatures, the percent extract weights were lower than the room temperature condition samples. For P1, the percent extract weights were about 10% to 33% (shaking or no-shaking, and 18G or 22 G syringe needles); for P2, the percent extract weights were about 22% to 41%; and for P3, the percent extract weights were about 20% to 66%.

For ground tablets at RT, the extract weights for P1, P2, and P3 decreased dramatically compared to intact tablets to only about 0% to 4% (shaking or no-shaking, and 18G or 22G syringe needles). At elevated temperature, the extract weights for P1, P2, and P3 increased relative to the RT samples, and were about 3% to 12%, about 20% to 37%, and about 5% to 28% for P1, P2, and P3, respectively.

α-6-mPEG₆-O-oxycodol Recovery: For ground low viscosity formulation tablets, under all conditions, about 67% to 77% of the α-6-mPEG₆-O-oxycodol content was recovered in the syringed fractions. For intact low viscosity formulation tablets, the recoveries were lower, at about 10% to 45%. Higher recoveries were observed for agitated samples, compared to no-shaking (non-agitated) samples. For intact tablets, α-6-mPEG₆-O-oxycodol recovery of the elevated temperature samples was higher compared to room temperature samples, but no apparent difference was seen for ground tablets.

α-6-mPEG₆-O-oxycodol recovery from high viscosity formulations P1, P2, and P3 was significantly lower at all the conditions tested. In general, room temperature condition samples showed lower α-6-mPEG₆-O-oxycodol recoveries compared to elevated temperature samples. There was no α-6-mPEG₆-O-oxycodol recovered from the ground P1, P2, and P3 formulations at room temperature. At elevated temperature (90° C.), maximum recoveries were about 10%, 31% and 22% for ground P1, P2, and P3, respectively. For intact P1, P2, and P3 formulations at room temperature, maximum recoveries were less than 10% (about 4%, 5%, and 6% for P1, P2, and P3, respectively), whereas at elevated temperature, maximum recoveries were about 13%, 18%, and 15% for P1, P2, and P3, respectively.

Conclusions

The low viscosity formulation was the most syringe-able, while the high viscosity P1 formulation was the least syringe-able. For all formulations, extract weights for intact tablets were generally higher but the α-6-mPEG₆-O-oxycodol recoveries were lower than for ground tablets. The α-6-mPEG₆-O-oxycodol recoveries for the high viscosity P1, P2, and P3 formulations were significantly lower than for the low viscosity formulation.

### Example 8

### Pharmacokinetics Study

The pharmacokinetic profiles of high viscosity formulations with varying NaCMC content (7.5% NaCMC or 10% NaCMC) in a fed state or a fasted state was compared to a reference non-high viscosity formulation (Formulation A) in a fed state or a fasted state. The pharmacokinetic study was conducted in healthy adult human subjects generally as described in Example 6.

Subjects were administered a single dose of one of compounds A, B, or C as provided in the table below.

Formulation A: Non-High Viscosity Formulation, 200 mg Tablet

| | Amount |
|---|---|
| Ingredient | Wt % |
| α-6-mPEGₙ-O-oxycodol phosphate | 29 |

Formulation B: High Viscosity Formulation, 200 mg Tablet

| | Amount |
|---|---|
| Ingredient | Wt % |
| α-6-mPEGₙ-O-oxycodol phosphate | 29 |
| NaCMC | 7.5 |

Formulation C: High Viscosity Formulation, 200 mg Tablet

| | Amount |
|---|---|
| Ingredient | Wt % |
| α-6-mPEGₙ-O-oxycodol phosphate | 29 |
| NaCMC | 10 |

The fed subjects fasted overnight for at least 10 hours and were then fed a high-fat meal 30 minutes prior to dosing. One of Formulation A, B or C was administered orally 30 minutes after the start of the meal. Subjects then fasted for at least four hours post dosing.

The fasted subjects fasted overnight for at least 10 hours prior to dosing and continued to fast for at least four hours post dosing.

Blood samples were collected at multiple time points through 72 hours post-dose. A linear mixed-effect model with treatment, period, and sequence as fixed effects, and subject nested in sequence as a random effect was fitted to logarithm-transformed Cₘₐₓ, AUC₀₋ₗₐₛₜ, and AUC_{0-inf} values to allow comparison of least squares (LS) means between administration in the fed and fasted states.

Following single oral doses of 7.5% or 10% NaCMC tablets under fed or fasted conditions, the active agent was readily absorbed with mean Tₘₐₓ values occurring between 2.3 to 3.1 hours across groups. Mean values for Cₘₐₓ, AUC₀₋ₗₐₛₜ, and AUC_{0-inf} were similar across all treatment groups. The mean t_{1/2} was comparable across all treatment groups and ranged from 10 to 11 hours.

The 90% CI for the geometric LS mean ratios of the fed state were within 80% to 125% of the fasted state for Cₘₐₓ, AUC₀₋ₗₐₛₜ, and AUC_{0-inf}, indicating that administration of the 7.5% and 10% NaCMC formulations were not affected by food intake.

### Example 9

### Pharmacokinetic Study

The pharmacokinetic profiles of high viscosity tablet formulations prepared with varying NaCMC content (7.5% NaCMC or 10% NaCMC), 10% xanthan gum, or a combination of 3.75% xanthan gum and 7.5% NaCMC by weight of the composition as compared to a non-high viscosity tablet formulation comprising α-6-mPEGₙ-O-oxycodol phosphate.

The pharmacokinetic study was conducted in healthy adult human subjects generally as described in Example 6. Subjects were administered a single dose of one of compounds A: 232.48 mg (equivalent to 200 mg) of α-6-mPEGₙ-O-oxycodol phosphate and 10% NaCMC; B: 232.48 mg (equivalent to 200 mg) of α-6-mPEGₙ-O-oxycodol phosphate and 10.6% xanthan gum, C: 200 mg 232.48 mg (equivalent to 200 mg) of α-6-mPEGₙ-O-oxycodol phosphate and 3.75% xanthan gum and 7.5% NaCMC; D: 200 mg 232.48 mg (equivalent to 200 mg) of α-6-mPEGₙ-O-oxycodol phosphate, REF. In the subject formulations, n=6.

Subjects were randomized to one of four different treatment sequences of 200 mg of A, B, C, and D based on a William's design consisting of two Latin squares. There were seven days between dose administrations. Each dose was administered after an overnight fast of at least 10 hours and the subjects remained fasted for at least 4 hours after dose administration. Blood samples were obtained pre-dose and at multiple time points over 72-hours post-dose.

The blood samples were assayed for the concentration of α-6-mPEG₆-O-oxycodol. The rate (peak concentration, Cₘₐₓ), time to maximum observed plasma drug concentration (Tₘₐₓ), and extent of absorption (area under the concentration-time curve, AUCₗₐₛₜ and AUC) were calculated for each of treatments A, B, C, and D.

The 90% CI of the geometric LS mean ratios between treatments (A, B and C) and the reference treatment D were within the 0.80 and 1.25 bioequivalence interval, with a geometric LS mean ratio of 1. The 90% CI for the ratio of geometric LS mean Cₘₐₓ between the 10% NaCMC formulation and the reference formulation fell within the bioequivalence interval of 0.80 and 1.25, with a geometric LS mean ratio of 0.9575 (90% CI, (0.8657, 1.0591)). In contrast, the 90% CI for the geometric LS mean ratio of Cₘₐₓ for formulations B and C (10.6% xanthan gum and combination xanthan gum/NaCMC) in comparison to formulation D was outside of the bioequivalence interval, with geometric LS mean ratios of 0.6651 (90% CI (0.6006, 0.7366) for formulation B and 0.7875 (90% CI (0.7120, 0.8710) for formulation C).

### Example 10

### Dissolution Study

Tablets comprised of Formulation A1, A4 or REF were tested for dissolution using USP<711> using Apparatus 2 (paddles at 75 rpm) in 900 mL of 01N HCl at 37.0±0.5° C. Samples were assayed for percentage of drug released by HPLC using an Agilent® HPLC ZORBAX® 300SB-C18 column, 3.5 μm, 4.6×50 nm with isocratic elution. Quantitation used a standard solution containing approximately 0.22 mg/mL of REF (as a free base). Dissolution profiles were prepared for Formulations A1, A4 and REF with the results provided in the table below and in Figure. 2.

| | | | Formulation A1 | | Formulation A4 | |
|---|---|---|---|---|---|---|
| | REF Formulation | | (10% NaCMC) | | (7.5% NaCMC) | |
| Time | % drug | | % drug | | % drug | |
| (min) | released | RSD | released | RSD | released | RSD |
| 5 | NT | NT | 17.7 | 23.1 | 21.9 | 16.3 |
| 10 | NT | NT | 57.8 | 9.2 | 64.0 | 2.1 |
| 15 | 58.8 | 11.2 | 90.6 | 5.8 | 93.7 | 1.1 |
| 30 | 100.7 | 2.6 | 102.1 | 1.4 | 101.0 | 1.0 |
| 45 | 104.7 | 1.5 | 101.6 | 1.4 | 101.7 | 1.1 |
| 60 | 104.8 | 1.5 | 101.8 | 1.1 | 101.1 | 1.0 |
| 120 | 104.9 | 1.6 | NT | NT | NT | NT |
| 180 | 104.5 | 1.3 | NT | NT | NT | NT |
| 240 | 104.7 | 1.7 | NT | NT | NT | NT |

As can be seen from the above, formulations A1 and A4 as well as the REF formulation all had complete (100%) release of the drug after 30 minutes. The formulations comprising NaCMC had at least 90% release of the drug after 15 minutes.

### INCORPORATION BY REFERENCE

All articles, books, patents, patent publications and other publications referenced herein are incorporated by reference in their entireties. In the event of an inconsistency between the teachings of this specification and the art incorporated by reference, the meaning of the teachings and definitions in this specification shall prevail (particularly with respect to terms used in the claims appended herein). For example, where the present application and a publication incorporated by reference defines the same term differently, the definition of the term shall be preserved within the teachings of the document from which the definition is located.

Exemplary Embodiments include the following:

1. An oral tablet high viscosity pharmaceutical composition comprising:
an opioid drug or a pharmaceutically acceptable salt thereof; and
a high viscosity agent;
wherein the weight percentage of the high viscosity agent is less than the weight percentage of the opioid drug in the composition.
2. The oral tablet high viscosity pharmaceutical composition of embodiment 1, wherein the high viscosity agent is PEG 3350, PEO (e.g., POLYOX™ WSR 308, POLYOX™ WSR Coagulant, POLYOX™ WSR 1105), HPMC (e.g., K100M), sodium alginate (e.g., PROTANAL® PH 6160, xanthan gum (e.g., XANTURAL® 75, XANTURAL® 180), carrageenan (e.g., GELCARIN® GP 379), NaCMC, (carrageenan), KLUCEL™ JXF Pharm, HPC (e.g., KLUCEL™ MF Pharm, KLUCEL™ MF), PEO 900K, PEO 400K, PEO (8 million), croscarmellose sodium (AC-DI-SOL®) xanthan gum, or a combination thereof.
3. The oral tablet high viscosity pharmaceutical composition of the combined or separate embodiments 1-2 as applicable, wherein the high viscosity agent is croscarmellose sodium (AC-DI-SOL®), NaCMC, xanthan gum, or a combination thereof.
4. The oral tablet high viscosity pharmaceutical composition of the combined or separate embodiments 1-3 as applicable, wherein the high viscosity agent is croscarmellose sodium (AC-DI-SOL®) and NaCMC.
5. The oral tablet high viscosity pharmaceutical composition of the combined or separate embodiments 1-3, wherein the high viscosity agent is NaCMC and Xanthan Gum.
6. The oral tablet high viscosity pharmaceutical composition of the combined or separate embodiments 1-3, wherein the high viscosity agent is croscarmellose sodium (AC-DI-SOL®) and Xanthan Gum.
7. The oral tablet high viscosity pharmaceutical composition of the combined or separate embodiments 1-3, wherein the high viscosity agent is NaCMC.
8. The oral tablet high viscosity pharmaceutical composition of the combined or separate embodiments 1-7, wherein the high viscosity agent is provided as an extra-granular component of the oral tablet high viscosity pharmaceutical composition.
9. The oral tablet high viscosity pharmaceutical composition of the combined or separate embodiments 1-7, wherein the high viscosity agent is provided as an intra-granular component of the oral tablet high viscosity pharmaceutical composition.
10. The oral tablet high viscosity pharmaceutical composition of the combined or separate embodiments 1-7, wherein the high viscosity agent is provided as both an extra-granular and an intra-granular component of the oral tablet high viscosity pharmaceutical composition.
11. The oral tablet high viscosity pharmaceutical composition of the combined or separate embodiments 1-10, wherein the high viscosity agent is from 5% to 50% of the total weight of the oral tablet high viscosity pharmaceutical composition.
12. The oral tablet high viscosity pharmaceutical composition of the combined or separate embodiments 1-10, wherein the high viscosity agent is from 5% to 15% of the total weight of the oral tablet high viscosity pharmaceutical composition.
13. The oral tablet high viscosity pharmaceutical composition of the combined or separate embodiments 1-10, wherein the high viscosity agent is from 7% to 12% of the total weight of the oral tablet high viscosity pharmaceutical composition.
14. The oral tablet high viscosity pharmaceutical composition of the combined or separate embodiments 1-10, wherein the high viscosity agent is about 7.5% the total weight of the oral tablet high viscosity pharmaceutical composition.
15. The oral tablet high viscosity pharmaceutical composition of the combined or separate embodiments 1-10, wherein the high viscosity agent is about 8.5% the total weight of the oral tablet high viscosity pharmaceutical composition.
16. The oral tablet high viscosity pharmaceutical composition of the combined or separate embodiments 1-15, wherein the opioid drug is according to Formula I:

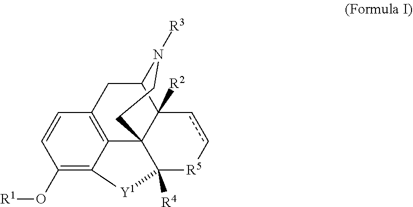

or a pharmaceutically acceptable salt, solvate, or prodrug thereof, wherein:
R¹ is hydrogen, —C(O)(C1-C10 alkyl), or C1-C10 alkyl;
R² is hydrogen or hydroxyl;
R³ is hydrogen or C1-C10 alkyl;
R⁴ is hydrogen or C1-C10 alkyl;

### Y¹ is —O— or —S—;

R⁵ is —C(O)— or —CH(OR⁶)—;
R⁶ is hydrogen, C1-C10 alkyl, —C(O)(C1-C10 alkyl), or —(CH₂CH₂O)ₙE¹;
n is a positive integer selected over the range of 1 to 30;
E¹ is hydrogen, methyl, or hydroxyl; and
the dotted line (---) represents an optional double bond.
17. The oral tablet high viscosity pharmaceutical composition of the combined or separate embodiments 1-15, wherein the opioid drug is according to Formula II:

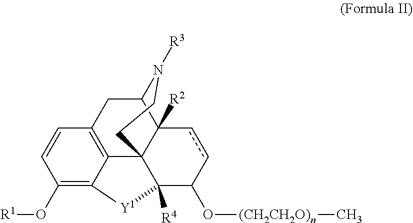

or a pharmaceutically acceptable salt, solvate, or prodrug thereof, wherein:
R¹ is hydrogen, —C(O)(C1-C10 alkyl), or C1-C10 alkyl;
R² is hydrogen or hydroxyl;
R³ is hydrogen or C1-C10 alkyl;
R⁴ is hydrogen or C1-C10 alkyl;

### Y¹ is —O— or —S—;

n is a positive integer selected over the range of 1 to 30; and
the dotted line (---) represents an optional double bond.
18. The oral tablet high viscosity pharmaceutical composition of the combined or separate embodiments 1-15, wherein the opioid drug is acetorphine, acetyldihydrocodeine, acetyldihydrocodeinone, acetylmorphinone, alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, etorphine, dihydroetorphine, fentanyl, heroin, hydrocodone, hydroxycodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphine, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propoxyphene, sufentanil, tilidine, tramadol, or a pharmaceutically acceptable salt thereof.
19. The oral tablet high viscosity pharmaceutical composition of the combined or separate embodiments 1-15, wherein the opioid drug is hydrocodone, morphine, hydromorphone, oxycodone, codeine, levorphanol, meperidine, methadone, oxymorphone, buprenorphine, fentanyl, dipipanone, heroin, tramadol, nalbuphine, etorphine, dihydroetorphine, butorphanol, levorphanol, or a pharmaceutically acceptable salt thereof.
20. The oral tablet high viscosity pharmaceutical composition of the combined or separate embodiments 1-15, wherein the opioid drug is α-6-mPEG₁₋₃₀-O-oxycodol, or more particularly, is α-6-mPEG₆-O-oxycodol, or a pharmaceutically acceptable salt thereof.
21. The oral tablet high viscosity pharmaceutical composition of the combined or separate embodiments 1-20, wherein the pharmaceutically acceptable salt of the opioid drug is an acid addition salt.
22. The oral tablet high viscosity pharmaceutical composition of the combined or separate embodiments 1-20, wherein the pharmaceutically acceptable salt of the opioid drug is a sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, gamma hydroxybutyrate, glycolate, tartrate, methanesulfonate, propanesulfonate, naphthalene 1-sulfonate, naphthalene-2-sulfonate, or mandelate salt, or mixture thereof.
23. The oral tablet high viscosity pharmaceutical composition of the combined or separate embodiments 1-20, wherein the pharmaceutically acceptable salt of the opioid drug is a base addition salt.
24. The oral tablet high viscosity pharmaceutical composition of the combined or separate embodiments 1-20, wherein the pharmaceutically acceptable salt of the opioid drug is an ammonium hydroxide, alkali hydroxide, alkaline earth metal hydroxide, carbonate, or bicarbonate base addition salt, or mixture thereof.
25. The oral tablet high viscosity pharmaceutical composition of the combined or separate embodiments 1-20, wherein the pharmaceutically acceptable salt of the opioid drug is a sodium hydroxide, potassium hydroxide, ammonium hydroxide, or potassium carbonate base addition salt, or mixture thereof.
26. The oral tablet high viscosity pharmaceutical composition of the combined or separate embodiments 1-15, wherein the opioid drug is α-6-mPEG₆-O-oxycodol phosphate salt or α-6-mPEG₆-O-oxycodol D-tartrate salt.
27. The oral tablet high viscosity pharmaceutical composition of the combined or separate embodiments 1-26, wherein the opioid drug is α-6-mPEG₆-O-oxycodol phosphate salt.
28. The oral tablet high viscosity pharmaceutical composition of the combined or separate embodiments 1-26, wherein the opioid drug is α-6-mPEG₆-O-oxycodol D-tartrate salt.
29. The oral tablet high viscosity pharmaceutical composition of the combined or separate embodiments 1-28, wherein the weight of the opioid drug is from 25% to 65% of the total weight of the oral tablet high viscosity pharmaceutical composition.
30. An oral tablet high viscosity pharmaceutical composition comprising: 28-30% by weight α-6-mPEG₆-O-oxycodol phosphate; and 6.5-11% by weight sodium carboxymethyl cellulose.
31. An oral tablet high viscosity pharmaceutical composition comprising: 28-30% by weight α-6-mPEG₆-O-oxycodol phosphate; and 6.5-8.5% by weight sodium carboxymethyl cellulose.
32. An oral tablet high viscosity pharmaceutical composition comprising: 28-30% by weight α-6-mPEG₆-O-oxycodol phosphate; and 7-8% by weight sodium carboxymethyl cellulose.
33. An oral tablet high viscosity pharmaceutical composition comprising:
(i) 28-30% by weight α-6-mPEG₆-O-oxycodol phosphate; and 9.5-10.5% by weight sodium carboxymethyl cellulose; or
(ii) 28-30% by weight α-6-mPEG₆-O-oxycodol phosphate; and 9.5-11.5% by weight xanthan gum; or
(iii) 28-30% by weight α-6-mPEG₆-O-oxycodol phosphate; 6.5-8.5% by weight carboxymethyl cellulose; and 0.5-2.0% by weight xanthan gum.
34. The oral tablet high viscosity pharmaceutical composition of any of the combined or separate embodiments 1-33 for use in the manufacture of a medicament for the treatment of pain.
35. The oral tablet high viscosity pharmaceutical composition of any of the combined or separate embodiments 1-33 for use in the treatment of pain.
36. A method of treating pain in a patient comprising administering a therapeutic amount of the oral tablet high viscosity pharmaceutical composition of any of the combined or separate embodiments 1-33 to the patient.
37. A solid composition comprising:

an α-6-mPEGₙ-O-oxycodol opioid drug, wherein n is an integer selected from 1 to 30, or a pharmaceutically acceptable salt thereof; and

at least one high viscosity agent;

wherein the composition when dissolved in an aqueous or alcoholic solution has a viscosity at 25° C. that is unsuitable for parenteral administration.

38. The composition of embodiment 37, wherein the viscosity of the composition is about 5-200 cP at 25° C. in an aqueous solution.
39. The composition of the combined or separate embodiments 37-38, wherein the viscosity of the composition is selected from at least 10 cP, at least 25 cP, at least 50 cP, at least 60 cP, at least 75 cP, at least 100 cP, at least 200 cP, at least 250 cP, at least 500 cP, at least 1000 cP, at least 1200 cP, at least 1500 cP, and about 1200-1600 cP for a 1% w/v aqueous solution at 25° C.
40. The composition of the combined or separate embodiments 37-39, wherein the at least one high viscosity agent comprises sodium carboxymethylcellulose (NaCMC).
41. The composition of the combined or separate embodiments 37-40, wherein the NaCMC has a degree of substitution selected from 0.65 to 1.45, 0.65 to 0.9, 0.80-0.95, 1.15-1.45, and at least about 0.65.
42. The composition of the combined or separate embodiments 37-41, wherein the NaCMC has a molecular weight of between 80,000 to 800,000 Da.
43. The composition of the combined or separate embodiments 37-42, wherein the composition comprises an amount of the high viscosity agent selected from 2.5-25%, 5 15%, 5-10%, 5-12%, 7.5-25%, 7.5-15%, 7.5-10%, 10-25%, 10-15%, 10-12%, and 12-15% of the high viscosity agent by weight.
44. The composition of the combined or separate embodiments 37-43, wherein the high viscosity agent is ionized at low pH less than 4.0; and wherein the high viscosity agent is unionized at a pH of 6.0-9.0.
45. The composition of the combined or separate embodiments 37-44, comprising a single high viscosity agent.
46. The composition of the combined or separate embodiments 37-45, wherein the opioid drug is α-6-mPEGₙ-O-oxycodol, wherein n is an integer selected from 1 to 10, or a pharmaceutically acceptable salt thereof.
47. The composition of the combined or separate embodiments 37-46, wherein the opioid drug has a molecular weight of 390 to 786 g/mol.
48. The composition of the combined or separate embodiments 37-47, comprising an amount of the opioid drug selected from 25-65% and 28-30% by weight of the composition.
49. The composition of the combined or separate embodiments 37-48, wherein the composition forms a gel when dissolved in an aqueous solution or an alcohol solution.
50. The composition of the combined or separate embodiments 37-49, for use in the treatment of pain.
51. A method of treating pain in a patient in need thereof, comprising administering a therapeutic amount of the composition of the combined or separate embodiments 37-50 to the patient.
52. The method of embodiment 51, wherein the composition is administered orally.
53. A solid dosage form comprising the composition of the combined or separate embodiments 37-50.
54. The dosage form of embodiment 53, wherein the solid dosage form is an oral dosage form.
55. The dosage form of the combined or separate embodiments 53-54, wherein the solid dosage form is a tablet or a capsule.
56. The solid dosage form of the combined or separate embodiments 53-55, further comprising a coating.

What is claimed is:

1. A solid composition comprising:
an α-6-mPEGₙ-O-oxycodol opioid drug, wherein n is an integer selected from 1 to 30, or a pharmaceutically acceptable salt thereof; and
at least one high viscosity agent;
wherein the composition when dissolved in an aqueous or alcoholic solution has a viscosity at 25° C. that is unsuitable for parenteral administration.

2. The composition of claim 1, wherein the viscosity of the composition is about 5-200 cP at 25° C. in an aqueous solution.

3. The composition of claim 1, wherein the viscosity of the composition is selected from at least 10 cP, at least 25 cP, at least 50 cP, at least 60 cP, at least 75 cP, at least 100 cP, at least 200 cP, at least 250 cP, at least 500 cP, at least 1000 cP, at least 1200 cP, at least 1500 cP, and about 1200-1600 cP for a 1% w/v aqueous solution at 25° C.

4. The composition of any one of claims 1-3, wherein the at least one high viscosity agent comprises sodium carboxymethylcellulose (NaCMC).

5. The composition of claim 4, wherein the NaCMC has a degree of substitution selected from 0.65 to 1.45, 0.65 to 0.9, 0.80-0.95, 1.15-1.45, and at least about 0.65.

6. The composition of any one of claims 1-5, wherein the NaCMC has a molecular weight of between 80,000 to 800,000 Da.

7. The composition of any one of claim 6, wherein the composition comprises an amount of the high viscosity agent selected from 2.5-25%, 5 15%, 5-10%, 5-12%, 7.5-25%, 7.5-15%, 7.5-10%, 10-25%, 10-15%, 10-12%, and 12-15% of the high viscosity agent by weight.

8. The composition of any one of claims 1-7, comprising a single high viscosity agent.

9. The composition of any one of claims 1-8, wherein the opioid drug is α-6-mPEGₙ-O-oxycodol, wherein n is an integer selected from 1 to 10, or a pharmaceutically acceptable salt thereof.

10. The composition of claim 9, wherein the opioid drug has a molecular weight of 390 to 786 g/mol.

11. The composition of claim 10, comprising an amount of the opioid drug selected from 25-65% and 28-30% by weight of the composition.

12. The composition of any one of claims 1-11, wherein the composition forms a gel when dissolved in an aqueous solution or an alcohol solution.

13. The composition of any one of claims 1-12 for use in the treatment of pain.

14. A method of treating pain in a patient in need thereof, comprising administering a therapeutic amount of the solid composition of any of claims 1-12 to the patient.

15. The method of claim 14, wherein the composition is administered orally.

16. A solid dosage form comprising the composition of any one of claims 1-13.

17. The dosage form of claim 16, wherein the solid dosage form is an oral dosage form.

18. The dosage form of any one of claims 16-17, wherein the solid dosage form is a tablet or a capsule.

19. The solid dosage form of any one of claims 16-18, further comprising a coating.

20. A method manufacturing a solid dosage form comprising:
mixing at least one opioid drug and at least one high viscosity agent;
forming the mixture into the solid dosage form;
wherein the solid dosage form, when dissolved in an aqueous or alcohol solution has a viscosity that is unsuitable for parenteral administration.
